(19) United States
(12) Patent Application Publication
(10) Pub. No.: US 2025/0177479 A1
(43) Pub. Date: Jun. 5, 2025

(54) DEPSIPEPTIDE-BASED AMPHIPHILIC BULIDING BLOCK TO INHIBIT PROTEIN-PROTEIN INTERACTIONS, NANOSTRUCTURE COMPRISED THEREOF AND USES THEREOF

(71) Applicant: INDUSTRY-ACADEMIC COOPERATION FOUNDATION, YONSEI UNIVERSITY, Seoul (KR)

(72) Inventors: Yong-Beom LIM, Seoul (KR); Hyunsuk HWANG, Yongin-si (KR)

(21) Appl. No.: 18/889,110

(22) Filed: Sep. 18, 2024

(30) Foreign Application Priority Data
Sep. 22, 2023 (KR) 10-2023-0127002

(51) Int. Cl. A61K 38/15; A61K 9/51; A61P 35/00; B82Y 5/00; C07K 11/00

(52) U.S. Cl. CPC A61K 38/15; A61K 9/5169; A61P 35/00; B82Y 5/00; C07K 11/00

(57) ABSTRACT

An embodiment relates to a depsipeptide-based building block for inhibiting protein-protein interactions, a nanostructure including the same, and a use thereof, wherein the depsipeptide-based building block may remain in the body and cells for a long time when administered in vivo and be delivered to a target tissue with high efficiency, and a peptide for inhibiting protein-protein interactions may be gradually released over a long time to obtain a high effect.

### CROSS-REFERENCE TO RELATED APPLICATION(S)

This application claims priority to Korean patent application No. 10-2023-0127002 filed on Sep. 22, 2023, in the Korean Intellectual Property Office, the entire disclosure of which is incorporated by reference herein.

### INCORPORATION OF SEQUENCE LISTING

This application contains a sequence listing in Extensible Markup Language (XML). The XML file containing the sequence listing entitled “9-PJK4967357-SequenceListing.xml”, which was created on Sep. 16, 2024, and is 29,468 bytes in size. The information in the sequence listing is incorporated herein by reference in entirety.

### BACKGROUND OF THE INVENTION

### Field of the Invention

The present invention relates to a depsipeptide-based building block for inhibiting protein-protein interactions (PPIs), a nanostructure including the same, and a use thereof. The present invention integrates a peptide drug into a depsipeptide-based building block that may be degraded by an esterase to provide a nanostructure and a pharmaceutical composition that have better biological activities and target site targeting efficiency and excellent pharmacokinetic properties compared to the conventional small molecule drugs.

### Description of the Related Art

Protein-protein interactions (PPIs) play an important role in the regulation of biological systems and are therefore a major target of drug development. Peptide or small molecule drugs have been developed as inhibitors for inhibiting PPIs. With the development of peptide library screening technology, peptide drugs have been identified and discovered quickly and easily. However, since they have poor in vivo pharmacokinetic properties and are unable to maintain their secondary structures, they have low actual in vivo activity, are unable to provide sufficient efficacy, and have ever-changing and unstable structures, making it impossible to control their pharmacokinetic properties.

Moreover, library screening only analyzes the inhibitory effect of PPIs in vivo, so the amount that is experimentally verified and registered does not exceed 0.4%. Therefore, despite numerous studies and efforts over the long history of peptide research, only a very small number of peptide drugs have been recognized as effective, and only an extremely small number have been approved for use. To date, peptide drugs approved for clinical applications are limited to hormones. This is due to the low pharmacokinetic properties of peptide drugs. For example, even when they exhibit excellent effects in vitro, they often fail to exhibit effects in cells or in vivo. This is because peptide drugs are rapidly hydrolyzed and easily eliminated in vivo, have low significantly low bioavailability due to their low cell membrane permeability, and are easily released from the body by the kidneys. Therefore, even when peptide drugs are developed, in order for them to be used as in vivo drugs, the development of a new platform for improving their pharmacokinetic properties is urgently needed.

Currently, most peptide drugs are delivered by encapsulation or entrapment in nanoparticles. However, the above-described methods have very low loading efficiency for peptide drugs, and there is a limitation in accumulating peptide drugs to target sites due to the formation of nanostructural aggregation between nanoparticles. Moreover, there has also been a problem that it is difficult to control the nanostructural properties or homogeneity during the nanoparticle preparation process. Accordingly, in the present invention, by using a nonpolar peptide drug as an important binding part of a self-assembled peptide structure, the present inventors made efforts to complete a new structural platform that can achieve 100% loading efficiency while achieving an anticancer effect superior to that of conventional anticancer agents through pharmacodynamic control, thereby completing the present invention.

The present invention is the result of the research project support described below.

1. [Research Project Number] 2400342811
[Detailed Project Number] RS-2024-00342811
[Research Management Organization] National Research Foundation of Korea
[Research Project Name] Magnetic Field Sensitivity, Structure, and Function Control of Biopolymer Assemblies
[Main Research Organization] Yonsei University Industry-Academic Foundation
[Research Period] May 1, 2024 to Apr. 30, 2027

2. [Research Project Number] 1711193996
[Detailed Project Number] 2022M3E5F101687721
[Research Management Organization] National Research Foundation of Korea
[Research Project Name] Development of Thermodynamically Stable and Safe Thread-Shaped mRNA Delivery Vehicle
[Main Research Organization] Yonsei University Industry-Academic Foundation
[Research Period] Apr. 1, 2022 to Dec. 31, 2025

3. [Research Project Number] 1711189750
[Detailed Project Number] 2022M3H4A1A02046445
[Research Management Organization] National Research Foundation of Korea
[Research Project Name] Development of Infrared Emissivity Control Materials for Energy Saving in Daily Living Environments
[Main Research Organization] Yonsei University Industry-Academic Foundation
[Research Period] Apr. 21, 2022 to Dec. 31, 2026

### RELATED ART DOCUMENTS

### [Patent Document]

(Patent document D1) Patent document D1: Korean Patent Publication 10-2018-0100967

### SUMMARY OF THE INVENTION

The present invention has been invented in consideration of the above-described problems, and an object of the present invention is to provide a peptide for controlling drug release for a cancer target represented by any one selected from the group consisting SEQ ID NOs: 1 to 4.

Another object of the present invention is to provide a depsipeptide-based building block including the peptide for controlling drug release.

Still another object of the present invention is to provide a spherical nanostructure having a bilayer membrane formed through self-assembly including the depsipeptide-based building block.

Yet another object of the present invention is to provide a pharmaceutical composition including the nanostructure.

To achieve the above-described objects, the present invention provides a peptide for controlling drug release for a cancer target, the peptide represented by any one selected from the group consisting of SEQ ID NOs: 1 to 4, wherein hydrogen positioned at a hydroxyl group at the C-terminus of the peptide is substituted with a carboxyl group having 1 to 5 carbon atoms.

The peptide for controlling drug release may have hydrophobicity and a function of inhibiting deformation of a secondary structure when combined with a peptide having an α-helix secondary structure.

To achieve the other object, the present invention provides depsipeptide-based building block consisting of: (a) the peptide for controlling drug release; (b) a peptide for inhibiting protein-protein interactions (PPIs) with an α-helix secondary structure consisting of 12 amino acid residues; and (c) a cell-penetrating peptide.

A C-terminal of the peptide for controlling drug release and an N-terminal of the peptide for inhibiting PPIs may be connected by an ester bond (—C(O)O—).

The (b) peptide for inhibiting PPIs may be a peptide having an activity of inhibiting the interaction between mouse double minute 2 homolog (MDM2) protein or murine double minute X homolog (MDMX) and p53 protein.

The (b) peptide for inhibiting PPIs may be a fragment of an α-helix peptide of p53 represented by SEQ ID NO: 13.

The (b) peptide for inhibiting PPIs may be any one selected from SEQ ID NOs: 14 to 18.

The (c) cell-penetrating peptide may be linked to a C-terminus of (b) the peptide for inhibiting PPIs by a peptide bond.

The cell penetrating peptide may be represented by any one selected from the group consisting of SEQ ID NOs: 19 to 21.

The total length of the depsipeptide-based building block may be 7 to 9 nm on average.

The (b) peptide for inhibiting PPIs and the (c) cell-penetrating peptide may be linked via a linker.

The linker may be one or more selected from Gly, Gly-Gly, and Gly-Gly-Gly.

To achieve the other object, the present invention provides a nanostructure of a spherical shape having a bilayer membrane formed through self-assembly, including the depsipeptide-based building block.

The depsipeptide-based building block may be any one of the depsipeptide-based building blocks represented by SEQ ID NOs: 5 to 12, or a mixture thereof.

The depsipeptide-based building block mixture may be a mixture of a depsipeptide-based building block represented by SEQ ID NO: 5 and a depsipeptide-based building block represented by SEQ ID NO: 6 at a molar ratio of 6:4.

The average diameter of the nanostructure may be 100 to 500 nm.

To achieve the other object, the present invention provides a pharmaceutical composition for treating or preventing cancer, including the nanostructure as an active ingredient

The cancer may be one or more selected from the group consisting of brain tumor, head and neck cancer, breast cancer, lung cancer, esophageal cancer, stomach cancer, duodenal cancer, appendix cancer, colon cancer, rectal cancer, liver cancer, pancreatic cancer, gallbladder cancer, bile duct cancer, anal cancer, kidney cancer, ureter cancer, bladder cancer, prostate cancer, penile cancer, testicular cancer, uterine cancer, ovarian cancer, vulvar cancer, vaginal cancer, or skin cancer.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 illustrates the structure of the peptide-based building blocks according to the present invention and a nanostructure formed through self-assembly thereof;

FIG. 2A shows a diagram illustrating in detail the structure of the peptide-based building blocks according to the present invention and the characteristics and roles of each component.

FIG. 2B shows a diagram illustrating the sequences and chemical structures of the peptide-based building blocks according to the present invention.

FIG. 3 shows a matrix-assisted laser desorption/ionization time-of-flight mass spectrometry (MALDI-TOF MS) spectrum of purified peptide-based building blocks: FIG. 3A shows a MALDI-TOF MS spectrum of a depsipeptide-based building block of Comparative Preparation Example 1; FIG. 3B shows a MALDI-TOF MS spectrum of a depsipeptide-based building block of Preparation Example 1; FIG. 3C shows a MALDI-TOF MS spectrum of a depsipeptide-based building block of Preparation Example 2; FIG. 3D shows a MALDI-TOF MS spectrum of a depsipeptide-based building block of Preparation Example 3; and FIG. 3E shows a MALDI-TOF MS spectrum of a depsipeptide-based building block of Preparation Example 4;

FIG. 4. shows a MALDI-TOF MS spectrum of a depsipeptide-based building block of Preparation Example 5;

FIG. 5A shows a MALDI-TOF MS spectrum of a depsipeptide-based building block of Preparation Example 7; and FIG. 5B shows a MALDI-TOF MS spectrum of a depsipeptide-based building block of Preparation Example 8;

FIG. 6A shows a MALDI-TOF MS spectrum of the depsipeptide-based building block of Preparation Example 9; and FIG. 6B shows a MALDI-TOF MS spectrum of the depsipeptide-based building block of Preparation Example 10;

FIG. 7 shows a high-performance liquid chromatography (HPLC) chromatogram of a purified peptide-based building block;

FIG. 8 shows a circular dichroism (CD) spectrum of the peptide-based building block (MIP-Tat) of Comparative Preparation Example 1;

FIG. 9 shows a CD spectrum of the depsipeptide-based building block (W5-MIP-Tat) of Preparation Example 1;

FIG. 10 shows a graph of analyzing the viability of HCT116 cells for SdPN and Nutlin-3a prepared according to Example 1 at concentrations of 0, 5, 10, and 20 μM;

FIG. 11 shows a graph of analyzing the viability of HCT116 cells for SdPN and Nutlin-3a prepared according to Example 3 at concentrations of 0, 5, 10, and 20 μM;

FIG. 12 shows a graph of analyzing the viability of HCT116 cells for SdPN and Nutlin-3a prepared according to Example 3 at concentrations of 0, 10, 20, and 40 μM;

FIG. 13 shows a graph analyzing the viability of HCT116 cells for co-self-assembled SdPN (W5-MIP-Tat/W5-MIP-RGD=5:5) prepared according to Example 10, co-self-assembled SdPN (W5-dummy-Tat/W5-dummy-RGD=5:5) prepared according to Example 11, and Nutlin-3a at concentrations of 0, 5, 10, and 20 μM;

FIG. 14 shows a graph analyzing the viability of HCT116 cells for SdPN according to the sequence for drug release control;

FIG. 15 shows a graph analyzing the viability of HCT116 cells for co-self-assembled SdPN (W5-MIP-Tat/W5-MIP-RGD-7.5 to 5:5 to 2.5) of Examples 5 to 10;

FIG. 16 shows a graph analyzing the viability of HCT116 cells for SdPN according to the proportion of the depsipeptides of Preparation Examples 5 and 6;

FIG. 17 shows a graph illustrating the dynamic light scattering (DLS) analysis results of SdPN (6:4) prepared from Example 8;

FIG. 18 shows a graph illustrating the CD analysis results of SdPN (6:4) prepared from Example 8 under various temperature conditions;

FIG. 19 shows a graph analyzing the DLS analysis results of SdPN (6:4) prepared from Example 8 under various temperature conditions;

FIG. 20 shows a graph analyzing the DLS analysis results of SdPN (6:4) prepared from Example 8 according to various storage periods;

FIG. 21 shows an atomic force microscopy (AFM) image (5×5 μm in size) of SdPN (6:4) prepared from Example 8;

FIG. 22 shows a transmission electron microscopy (TEM) image of SdPN (6:4) prepared from Example 8;

FIG. 23 shows an AFM image of depsipeptide-based SdPN of Preparation Example 6;

FIG. 24 shows an AFM image of depsipeptide-based SdPN of Preparation Example 10;

FIG. 25 shows a fluorescence emission spectrum (λₑₓ=280 nm) of SdPN (6:4) prepared from Example 8;

FIG. 26 shows a graph analyzing the viability of HCT116 p53^{+/+} cells treated with SdPN (6:4) prepared from Example 8 and idasanutlin;

FIG. 27 shows the results of analyzing the reaction behavior (kinetic analysis) of SdPN (6:4) prepared from Example 8 and idasanutlin on MDM2 and p53 analyzed by immunoblotting;

FIG. 28 shows a graph illustrating the quantification of the p53 expression level from the immunoblotting results of FIG. 27;

FIG. 29 shows a graph illustrating the degree of ester bond degradation of depsipeptide-based building blocks (W5-MIP-Tat and W5-MIP-RGD) analyzed by HPLC when SdPN (6:4) prepared from Example 8 was incubated under conditions in which an esterase was absent;

FIG. 30 shows a graph illustrating the degree of ester bond degradation of depsipeptide-based building blocks (W5-MIP-Tat and W5-MIP-RGD) analyzed by HPLC when SdPN (6:4) prepared from Example 8 was incubated under esterase conditions of various concentrations;

FIG. 31 shows the results of a Caspase-3/7 activity analysis for SdPN (6:4) of Example 8, dummy-SdPN (6:4) of Example 12, and idasanutlin;

FIG. 32 shows the results of a Caspase-3/7 activity analysis for depsipeptide-based SdPN of Preparation Example 6 and depsipeptide-based SdPN of Preparation Examples 5 and 6 (1:1);

FIG. 33 shows the results of a cellular thermal shift assay (CETSA) for SdPN (6:4) of Example 8 and dummy-SdPN (6:4) of Example 11;

FIG. 34 shows a graph illustrating the quantitative analysis of the CETSA results for analyzing the Tm (melting temperature) of SdPN (6:4) of Example 8 and dummy-SdPN (6:4) of Example 11;

FIG. 35 shows the iso-CETSA results of SdPN (6:4) prepared from Example 8 and idasanutlin;

FIG. 36 shows a graph illustrating the IC50 level obtained from the iso-CETSA results of SdPN (6:4) prepared from Example 8 or idasanutlin;

FIG. 37 shows a graph illustrating the change in tumor volume of xenografted mice measured after the intravenous administration of SdPN (6:4) (experimental group) of Example 8, idasanutlin (positive control group), and saline (control group);

FIG. 38 shows a graph illustrating the tumor weight of xenografted mice 14 days after the intravenous administration of SdPN (6:4) (experimental group) of Example 8, idasanutlin (positive control group), and saline (control group);

FIG. 39 shows a graph illustrating the change in body weight of xenografted mice measured after the intravenous administration of SdPN (6:4) (experimental group) of Example 8, idasanutlin (positive control group), and saline (control group);

FIG. 40 shows the results of hematoxylin and eosin (H&E) staining of cancer tissues isolated from xenografted mice 14 days the intravenous administration of SdPN (6:4) (experimental group) of Example 8, idasanutlin (positive control group), and saline (control group) (bar=50 μm);

FIG. 41 shows the results of H&E staining of major organ tissues (heart, lung, liver, spleen, and kidney) isolated from xenografted mice 14 days after the intravenous administration of SdPN (6:4) (experimental group) of Example 8, idasanutlin (positive control group), and saline (control group) (bar=100 μm);

FIGS. 42A-42D respectively show the synthesis process (FIG. 42A), MALDI-TOF MS (FIG. 42B), fluorescence emission spectrum (FIG. 42C), and DLS analysis results (FIG. 42D) of SdPN (6:4) (SdPN-dye) of Example 8 labeled with sulfo-cyanine 5.5 (Cy5.5);

FIG. 43 shows in vivo fluorescence images (in vivo imaging system, IVIS) of xenografted mice treated with Cy5.5-labled SdPN (6:4) (SdPN-dye) of Example 8 and Cy5.5;

FIG. 44 shows a graph illustrating the fluorescence intensity quantified from the IVIS image data of FIG. 43 (mean±standard deviation, n=4, ***P<0.001);

FIG. 45 shows a graph illustrating the fluorescence intensity measured in the blood of xenografted mice treated with Cy5.5-labled SdPN (6:4) (SdPN-dye) of Example 8 and Cy5.5;

FIG. 46 shows fluorescence images of frozen sections of cancer tissues isolated from xenografted mice 24 hours after the intravenous administration of Cy5.5-labled SdPN (6:4) (SdPN-dye) of Example 8 and Cy5.5 (bar=50 μm);

FIG. 47 shows a fluorescence image of tail vein blood obtained from xenografted mice intravenously administered with Cy5.5-labled SdPN (6:4) (SdPN-dye) of Example 8 and Cy5.5 (bar=50 μm);

FIG. 48 shows fluorescence images of major organs (heart, lung, liver, spleen, and kidney) and cancer tissues isolated from a xenograft mouse 24 hours after the intravenous administration of Cy5.5-labled SdPN (6:4) (SdPN-dye) of Example 8 and Cy5.5;

FIG. 49 shows a graph the fluorescence intensity quantified from the fluorescence image data of FIG. 48; and

FIG. 50 illustrates the mechanism by which the depsipeptide-based nanostructure according to the present invention acts on cancer cells when injected into the body.

### DETAILED DESCRIPTION OF THE EMBODIMENTS

Hereinafter, various aspects and embodiments of the present invention will be described in more detail.

The objects, other objects, features, and advantages of the present invention will be easily understood through the following preferred embodiments in conjunction with the accompanying drawings. However, the present invention is not limited to the embodiments described herein and may be embodied in other forms. Rather, the embodiments introduced herein are provided so that the disclosed content may become thorough and complete and so that the spirit of the present invention can be sufficiently conveyed to those skilled in the art.

In the present invention, terms such as “comprise” or “have” should be understood as specifying the presence of features, numbers, steps, operations, components, parts or combinations thereof described in the specification, not precluding the possibility of the presence or addition of one or more other features, numbers, steps, operations, components, parts or combinations thereof.

In the present specification, it will be understood that when a range is described for a variable, the variable includes all values within the described range including the described endpoints of the range. For example, a range of “5 to 10” will be understood to include the values 5, 6, 7, 8, 9, and 10, as well as any subranges of 6 to 10, 7 to 10, 6 to 9, 7 to 9, and the like, and also any values between the reasonable integers that fall within the described range, such as 5.5, 6.5, 7.5, 5.5 to 8.5, and 6.5 to 9. In addition, for example, a range of “10% to 30%” will be understood to include not only the values of 10%, 11%, 12%, 13%, and the like, and all integers up to and including 30%, but also any subranges of 10% to 15%, 12% to 18%, 20% to 30%, and the like and also any values between reasonable integers within the described range, such as 10.5%, 15.5%, 25.5%, and the like.

One aspect of the present invention provides a peptide for controlling drug release for a cancer target, the peptide being selected from the group consisting of SEQ ID NOs: 1 to 4.

| | |
|---|---|
| SEQ ID NO: 1 | WWWWW |
| SEQ ID NO: 2 | WKWE |
| SEQ ID NO: 3 | III |
| SEQ ID NO: 4 | IIII |

The peptide is characterized in that the hydrogen positioned at a hydroxyl group at the C-terminus is substituted with a carboxyl group having 1 to 5 carbon atoms, preferably substituted with a carboxyl group having 1 to 2 carbon atoms, and more preferably substituted with a carboxyl group having 2 carbon atoms.

In the present invention, a peptide means a linear molecule formed by amino acids being linked to each other by peptide bonds.

For reference, the representative amino acids and their respective abbreviations are: alanine (Ala, A), isoleucine (Ile, I), leucine (Leu, L), methionine (Met, M), phenylalanine (Phe, F), proline (Pro, P), tryptophan (Trp, W), valine (Val, V), asparagine (Asn, N), cysteine (Cys, C), glutamine (Gln, Q), glycine (Gly, G), serine (Ser, S), threonine (Thr, T), tyrosine (Tyr, Y), aspartic acid (Asp, D), glutamic acid (Glu, E), arginine (Arg, R), histidine (His, H), and lysine (Lys, K).

The peptide of the present invention may be prepared according to chemical synthesis methods known in the art, particularly, solid-phase synthesis techniques (Merrifield, J. Amer. Chem. Soc. 85:2149-54 (1963); Stewart et al., Solid Phase Peptide Synthesis, 2nd. ed., Pierce Chem. Co.: Rockford, 111 (1984)).

The peptide for controlling drug release according to the present invention contains a hydrophobic structure that binds to a peptide for inhibiting protein-protein interactions (PPIs) having an α-helix secondary structure consisting of 12 amino acid residues, so that when administered in vivo or stored in a solution and utilized as a nanostructure through self-assembly, the secondary structure of (b) the peptide for inhibiting PPIs may be fixed and maintained without being deformed, thereby inhibiting the loss or deterioration of biological activity.

In addition, when the hydrogen positioned at —OH of the C-terminus is substituted with a carboxyl group having 1 to 5 carbon atoms, the peptide for controlling drug release according to the present invention forms depsipeptide through an ester bond when combined with (b) the peptide for inhibiting PPIs having biological activity. Therefore, a peptide having biological activity may be loaded with high efficiency, there is no problem of aggregation in a solution. In addition, since the peptide is not sensitive to temperature, it easily penetrates cells in vivo, remains in vivo for a long time, and accumulates in cancer cells, In addition, the release of (b) the peptide for inhibiting PPIs in cells is gradually and continuously carried out, the pharmacodynamic properties may be easily controlled.

In addition, the peptide for controlling drug release according to the present invention is cleaved by an esterase, which exists in excess particularly in cancer cells, through an ester bond with a biologically active peptide (e.g., peptide (b) for inhibiting PPIs), so that (b) the peptide for inhibiting PPIs may be released to specifically act on cancer cells.

In addition, since the peptide for controlling drug release according to the present invention has strong hydrophobicity, it is located inside (hydrophobic region) of a bilayer membrane on a nanostructure, and thus has stability such that it is hardly decomposed under physiological conditions, that is, outside cells.

Accordingly, the peptide for controlling drug release according to the present invention is bonded to (b) the peptide for inhibiting PPIs through an ester bond, and since both the amino acid units of the polypeptide and the hydrophobic structure units are composed of safe substances, it has little or no toxicity due to the final decomposition products.

The method for preparing the peptide for controlling drug release according to the present invention is not particularly limited. For example, it may be prepared by preparing a peptide structure consisting of five tryptophan residues and then bonding a specific or arbitrary —COOH group of its amino acid residues with a carboxyl group having 1 to 5 carbon atoms. At this time, amide bonding may be performed by an organic synthesis reaction, a biosynthetic reaction using microorganisms, an enzymatic synthesis reaction using natural or synthetic enzymes, or the like, but is not limited to, and the bonding order of each site is not limited, either. In addition, instead of a peptide structure consisting of five tryptophan residues, a peptide structure consisting of (WKWE)2, III, or IIII may be prepared, and then it may be prepared using the same process as above.

Another aspect of the present invention relates to a depsipeptide-based building block consisting of: (a) the peptide for controlling drug release according to claim 1; (b) a peptide for inhibiting PPIs with an α-helix secondary structure consisting of 12 amino acid residues; and (c) a cell-penetrating peptide.

The depsipeptide-based building block is characterized in that a C-terminal of (a) the peptide for controlling drug release and an N-terminal of (b) the peptide for inhibiting PPIs are connected by an ester bond (—C(O)O—).

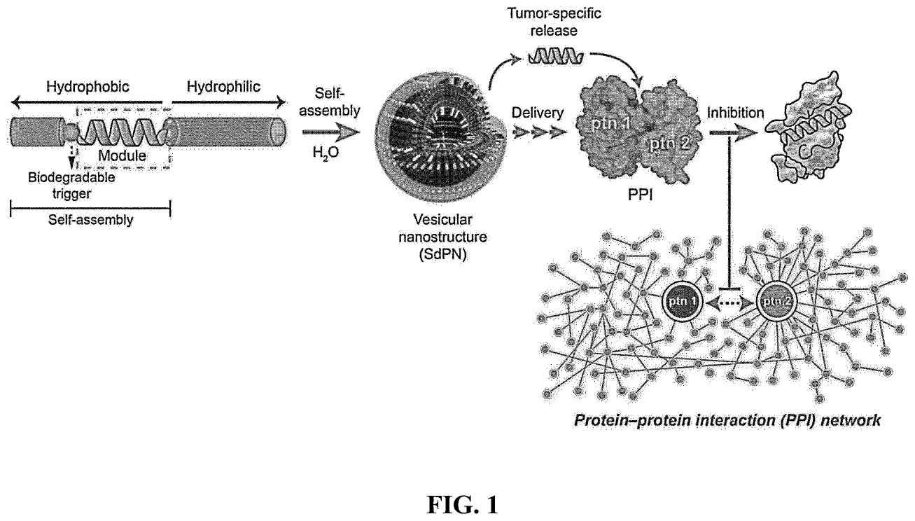

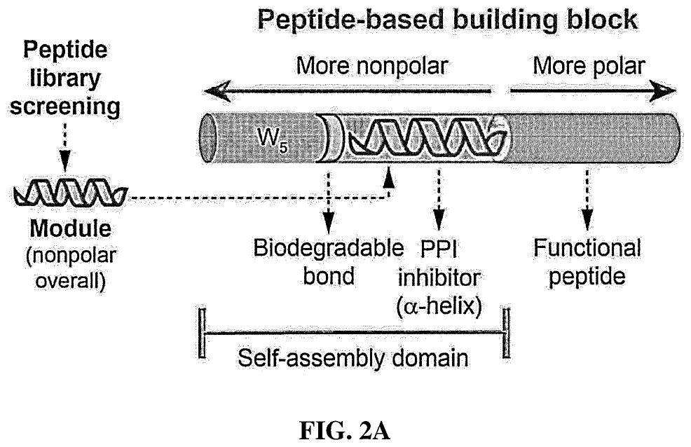

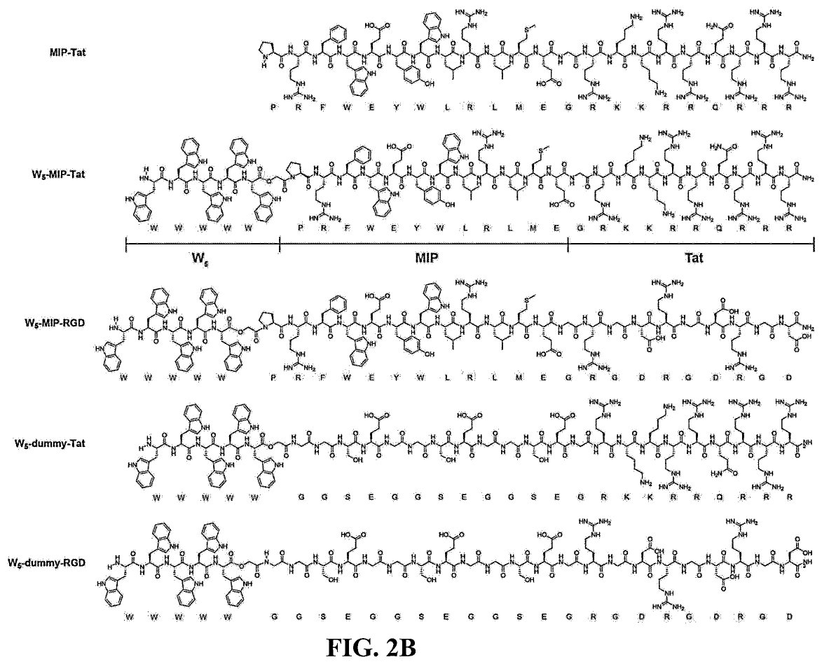

The structure of the depsipeptide-based building block according to the present invention is shown in FIG. 1 and FIG. 2A. FIG. 1 illustrates the structure of the peptide-based building blocks according to the present invention and a nanostructure formed through self-assembly thereof, and FIG. 2A shows a diagram illustrating in detail the structure of the peptide-based building blocks according to the present invention and the characteristics and roles of each component.

Referring to FIGS. 1 and 2A, the depsipeptide-based building block according to the present invention includes (a) a peptide for controlling drug release; (b) a peptide for inhibiting PPIs having an α-helical secondary structure consisting of 12 amino acid residues; and (c) a cell-penetrating peptide; and (a) the peptide for controlling drug release and (b) the peptide for inhibiting PPIs having an α-helical secondary structure consisting of 12 amino acid residues are linked by an ester bond (—C(O)O—) and have a function of being biodegraded by an esterase, which is present in a large amount particularly in cancer cells, after being delivered into cells (‘biodegradable bond’).

The depsipeptide-based building block according to the present invention is a linear depsipeptide having amphiphilicity, including the hydrophobic portions of (a) and (b) and the hydrophilic portion of (c), and acts as a building block that forms a spherical nanostructure having a bilayer membrane through self-assembly in a solution phase.

The (a) peptide for controlling drug release according to the present invention is identical to the “peptide for controlling drug release represented by any one selected from the group consisting of SEQ ID NOs: 1 to 4,” and therefore, to avoid repetition, it is omitted and the above contents are referred to. The (a) peptide for controlling drug release is a strongly nonpolar molecule.

The above (b) the peptide for inhibiting PPIs, which is a peptide having an α-helical secondary structure consisting of 12 amino acid residues, is not particularly limited to a specific amino acid sequence as long as it is a peptide having a biological activity of inhibiting PPIs. According to Experimental Example 2 of the present invention, it can be confirmed that the co-self-assembled SdPNs of Examples 11 and 12, which included a dummy sequence including a random sequence, were also efficiently delivered into cells when administered in vivo or to cells, and degraded by an esterase, and they successfully delivered the dummy peptide into cells. In addition, it was confirmed that the co-self-assembled SdPNs of Examples 11 and 12 also had thermal stability, and that their structures were maintained even when stored at high temperatures and for a long time. In other words, any peptide consisting of 12 amino acid residues having an α-helix structure may be used as (b) a peptide for inhibiting PPIs, and even when the sequence is different, the amphiphilicity of the depsipeptide-based building block according to the present invention, the formation of self-assembly, intracellular delivery, release rate, and the like are not affected, so that the purpose can be successfully achieved.

Since hydrophobic interactions are applied to PPIs, a large nonpolar interface is formed at the PPI interface, and most peptide drugs that inhibit PPIs contain many nonpolar residues. Since the α-helix structure is advantageous for specific binding in regulating PPIs, most peptide drugs have an α-helix structure.

The (b) peptide for inhibiting PPIs according to the present invention may provide a basis for forming a spherical nanostructure of a bilayer membrane through self-assembly in a solution phase while ensuring that the α-helix secondary structure is stably maintained without being unfolded due to (a) the peptide for controlling drug release.

According to one embodiment of the present invention, (b) the peptide for inhibiting PPIs may be a biomolecule that blocks disease-associated PPIs, thereby treating, preventing, or ameliorating the associated disease.

In the present invention, disease-associated PPIs means that various diseases are induced or caused by abnormal protein binding (homomeric, heteromeric), and various disease-associated PPI targeting substances are known through various protein analysis technologies such as co-immunoprecipitation (Co-IP), high-content screening (HCS), fluorescence resonance energy transfer (FRET), and high throughput screening (HTS).

Therefore, (b) the peptide for inhibiting PPIs is not particularly limited as long as it is a peptide that has the activity of disease-associated PPIs discovered by various protein analysis technologies such as Co-IP, HCS, FRET, mRNA display library technology, and HTS. However, (b) the peptide for inhibiting PPIs may achieve the role of inhibiting PPIs when about 50% to 75% of the residues among the 12 amino acid residues include one or more selected from nonpolar amino acid residues (Gly, Ala, Val, Leu, Ile, Met, Pro, Phe, Thr, Cys, Tyr, Trp).

More specifically, (b) the peptide for inhibiting PPIs may be a peptide having an activity of inhibiting PPIs associated with the growth or progression of cancer.

The (b) peptide for inhibiting PPIs may be a peptide having an activity of inhibiting the interaction between mouse double minute 2 homolog (MDM2) protein or murine double minute X homolog (MDMX) and p53 protein. Specifically, it may refer to a peptide that specifically binds to MDM2 protein or MDMX and inhibits the function of MDMs.

The (b) peptide for inhibiting PPIs serves as a competitive substrate for a protein (p53) that binds to MDM2 or MDMX, and thus has the effect of inhibiting the binding to various proteins that bind to MDM2 or MDMX.

The (b) peptide for inhibiting PPIs may be derived from an α-helix portion of p53 for binding to MDM2 or MDMX and p53, specifically, it may be a fragment of the α-helix peptide of p53 represented by SEQ ID NO: 13, and preferably any one selected from SEQ ID NOs: 14 to 18.

It was confirmed through a circular dichroism (CD) analysis that (b) the peptide for inhibiting PPIs binds to a depsipeptide having an ester bond through the binding to (a) the peptide for controlling drug release, and (b) the peptide for inhibiting PPIs is formed in an α-helix structure in this state.

The (c) cell-penetrating peptide is linked to a C-terminus of (b) the peptide for inhibiting PPIs by a peptide bond. The (c) cell-penetrating peptide is not particularly limited as long as it is a peptide having hydrophilic cell-penetrating activity, but may preferably be Tat or RGD, and these may be represented by SEQ ID NO: 19 or SEQ ID NO: 20. The cell-penetrating peptide represented by SEQ ID NO: 20 is an RGD peptide, which is convenient for active cancer targeting through an integrin receptor highly expressed in cancer cells. As another embodiment, the cell-penetrating peptide may be RRR of SEQ ID NO: 21.

The total length of the depsipeptide-based building block is preferably 7 to 9 nm on average.

The (b) peptide for inhibiting PPIs and (c) cell-penetrating peptide may further include a linker for connection. The linker may be one or more selected from Gly, Gly-Gly, and Gly-Gly-Gly. In order for the depsipeptide-based building block according to the present invention to obtain an excellent pharmaceutical effect by effectively releasing only (b) the peptide for inhibiting PPIs by degradation of the two peptides (b, c) by an esterase within a cell without interfering with the intracellular penetration and accumulation, the linker is preferably Gly.

Another aspect of the present invention relates to a spherical nanostructure having a bilayer membrane formed through self-assembly including the depsipeptide-based building block.

The nanostructure according to the present invention is a spherical vesicle-shaped nanostructure having a bilayer membrane, which is formed through self-assembly in a solution phase by the properties of the depsipeptide-based building block having the above-described structure. Specifically, a plurality of the depsipeptide-based building blocks form a bilayer membrane, and the bilayer membrane is connected in a spherical shape to form a spherical vesicle-shaped nanostructure.

In the depsipeptide-based building block, the hydrophobic portions (a, b) including (a) a peptide for controlling drug release and (b) a peptide for inhibiting PPIs are arranged toward the inside of the bilayer membrane, and the hydrophilic portion including (c) a cell-penetrating peptide is arranged toward the outside of the bilayer membrane. At this time, hydrophobic interactions are formed between the hydrophobic fragments (a, b), and thereby a plurality of depsipeptide-based building blocks may be combined to form a membrane having a “bilayer” structure. The bilayer membrane is manufactured as a spherical nanostructure, which may also be referred to as a “self-organized assembly.”

Due to the above-described structure, the nanostructure according to the present invention has excellent thermal stability such that the nanostructure structure does not unfold even at a temperature of 10 to 70° C., and has excellent structural stability such that the structure is deformed even when stored for a long time.

In addition, the nanostructure according to the present invention, when injected into a living body, is distributed throughout the body through the blood vessels, diffuses into tissues outside the blood vessels, passes through the cell membranes of each tissue, and efficiently accumulates within cells, and is degraded by an enzyme (esterase) present in specific cells or tissues to release the drug, so it not only has a specific pharmaceutical effect on the target site, but also has the advantages of a slow rate of elimination from cells or tissues, that is, the body (elimination half-life), a long drug effect onset time, and a long drug effect duration. Therefore, the nanostructure according to the present invention is pharmacodynamically designed so that after being injected into a living body, it sufficiently reaches each organ or cell, and then the efficacy is slowly exhibited in specific target cells and maintained for a long time, so it has the advantages of a small number of drug administration times and a uniform reaction, and it can be used as a therapeutic agent for various diseases.

The nanostructure according to the present invention may be easily administered when administered in combination with other drugs, in consideration of the drug effect onset time and the drug effect duration.

The efficacy of the nanostructure according to the present invention is due to the effect of (b) the peptide for inhibiting PPIs included in the depsipeptide-based building block, and since this is present in the bilayer film, an excellent pharmaceutical effect can be obtained with the nanostructure alone.

In addition, the depsipeptide-based building block forming the nanostructure according to the present invention may be any one of the depsipeptide-based building blocks represented by SEQ ID NO: 5 and SEQ ID NO: 6, or a mixture thereof, and preferably may be a mixture of the depsipeptide-based building block represented by SEQ ID NO: 5 and the depsipeptide-based building block represented by SEQ ID NO: 6. At this time, the depsipeptide-based building block represented by SEQ ID NO: 5 and the depsipeptide-based building block represented by SEQ ID NO: 6 differ in that they have different cell-penetrating peptides, and a nanostructure formed through their co-self-assembly can reduce the occurrence of side effects such as non-specific necrotic cytotoxicity and significantly increase the efficacy of (b) the peptide for inhibiting PPIs.

The mixture of the depsipeptide-based building blocks may be a mixture of the depsipeptide-based building block represented by SEQ ID NO: 5 and the depsipeptide-based building block represented by SEQ ID NO: 6 at a molar ratio of 8 to 5:2 to 5, preferably a mixture at a molar ratio of 5 to 6:4 to 5, and most preferably a mixture at a molar ratio of 6:4. In this case, the IC50 for cancer cells may be 1 to 5 μM, which may exhibit affinity for the target protein three times higher than that of conventional drugs, thereby exhibiting strong efficacy.

A water-soluble substance may be further included in the internal space (may also be referred to as the core) of the nanostructure according to the present invention. The water-soluble substance may be present in a form encapsulated in the nanostructure, thereby providing high functionality.

The water-soluble substance is not particularly limited as long as it is a water-soluble drug for providing the same or different efficacy to the nanostructure of the present invention, or a water-soluble substance for providing an additional function to the nanostructure of the present invention, and is also referred to as ‘cargo.’

The above water-soluble substance is not particularly limited, but may specifically include one or more selected from the group consisting of proteins, peptides, nucleic acid molecules, sugars, lipids, nanoparticles, compounds, and fluorescent substances, and preferably include one or more selected from the group consisting of compounds having antiviral, antimicrobial (antibiotic), and anticancer (chemotherapy) activity such as dexorubicin, paciltazole, mitomycin C, 17-AAG, velcade, phosphorylated nucleosides, phosphorylated peptides, and the like; experimental, therapeutic, or magnetic resonance imaging (MRI) reagents such as galdolium; radio-opaque drugs used in X-ray-based analysis; radioactive mixtures and reagents used in positron emission tomography (PET) scanning (e.g., carbon-11, nitrogen-13, oxygen-18, and fluorine-18); radioactive compounds and reagents used in single photon emission computed tomography (SPECT) scans (e.g., iodine-123, technetium-99, xenon-133, thallium-201, and fluorine-18); and DNA and RNA molecules and nucleic acids having single or double strands. The nucleic acids may include complementary DNA (cDNA), mitochondrial DNA, chloroplast DNA, anti-sense DNA, and small interfering RNA (siRNA) expressing a detectable protein.

In addition, the water-soluble substance may include water-soluble molecules that may be used for various biological effects or diagnostic purposes.

The average diameter of the nanostructures may be 100 to 500 nm.

In addition, since the nanostructure of the present invention is prepared through self-assembly in a solution phase such as saline solution or distilled water, unlike an oil-based emulsion method, it does not use a large amount of toxic organic solvent, and is therefore a biocompatible material with low toxicity even without undergoing a separate purification process.

Another aspect of the present invention relates to pharmaceutical composition for treating or preventing cancer, including the nanostructure as an active ingredient.

The pharmaceutical composition of the present invention may provide a pharmaceutically effective amount to an individual through various administration routes by any appropriate method.

The term ‘cancer’ used herein is a general term for a disease caused by cells that have an aggressive characteristic in which cells divide and grow while ignoring the normal growth limits, an invasive characteristic in which cells infiltrate surrounding tissues, and a metastatic characteristic in which cells spread to other parts of the body. The type of cancer is not particularly limited, but may be one or more selected from the group consisting of tumors, head and neck cancer, breast cancer, lung cancer, esophageal cancer, stomach cancer, duodenal cancer, appendix cancer, colon cancer, rectal cancer, liver cancer, pancreatic cancer, gallbladder cancer, bile duct cancer, anal cancer, renal cancer, ureteral cancer, bladder cancer, prostate cancer, penile cancer, testicular cancer, uterine cancer, ovarian cancer, vulvar cancer, vaginal cancer, and skin cancer. In one embodiment of the present invention, the anticancer effect was confirmed using HCT116, a colon cancer cell line, but is not limited thereto.

The term ‘treatment or prevention’ as used herein includes alleviating symptoms of a disease, condition, or disorder, or preventing or inhibiting further occurrence of the disease, condition, or disorder, by preventing or delaying the occurrence of symptoms, complications, or biochemical signs of the disease or disorder by administering the pharmaceutical composition of the present invention. The treatment may be prophylactic suppression (to prevent or delay the onset of a disease, or to prevent the manifestation of clinical or subclinical symptoms thereof) or therapeutic suppression or alleviation of symptoms after a disease has appeared.

As used herein, the term ‘prevent,’ ‘preventing,’ or ‘prevention’ in relation to a disease or disorder refers to prophylactically treating a subject at risk of developing a condition (e.g., a disease or disorder, or a specific disease or disorder such as cancer or a clinical symptom thereof) to reduce the probability of the subject developing the condition.

In the present invention, the pharmaceutical composition may further including an appropriate carrier, excipient, or diluent according to a conventional method. Carriers, excipients, and diluents that may be included in the pharmaceutical composition of the present invention include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, methylhydroxy benzoate, propylhydroxybenzoate, talc, magnesium stearate, and mineral oils, but are not limited thereto.

In addition, the pharmaceutical composition according to the present invention may be formulated and used in the form of oral dosage forms such as powder, granules, tablets, capsules, suspensions, emulsions, syrups, and aerosols, external preparations, suppositories, or sterile injection solutions according to respective conventional methods. Specifically, when formulated, the pharmaceutical composition according to the present invention may be prepared using diluents or excipients such as commonly used fillers, bulking agents, binders, wetting agents, disintegrants, and surfactants. Solid preparations for oral administration include tablets, pills, powder, granules, capsules, and the like, and such solid preparations may be prepared by mixing the pharmaceutical composition of the present invention with one or more excipients such as starch, calcium carbonate, sucrose, lactose, and gelatin. In addition to simple excipients, lubricants such as magnesium stearate and talc may also be used. Liquid preparations for oral administration include suspensions, solutions, emulsions, and syrups, and in addition to commonly used simple diluents such as water and liquid paraffin, they may contain various excipients such as wetting agents, sweeteners, flavoring agents, and preservatives. Preparations for parenteral administration include sterile aqueous solutions, non-aqueous solvents, suspensions, emulsions, lyophilized preparations, and suppositories. Non-aqueous solvents and suspending agents may include propylene glycol, polyethylene glycol, vegetable oils such as olive oil, and injectable esters such as ethyl oleate. As suppository bases, Witepsol, Macrogol, Tween 61, cacao butter, lauric butter, glycerogelatin, and the like may be used.

The term ‘individual’ used herein refers to a subject in need of treatment for a disease, and more specifically, it may be a mammal such as a human or non-human primate, mouse, rat, dog, cat, horse, or cow, but is not limited thereto.

In the pharmaceutical composition of the present invention, ‘administration’ refers to introducing a predetermined substance to a patient by any appropriate method, and the ‘administration route’ of the pharmaceutical composition refers to any general route through which a drug may reach a target tissue, and is not particularly limited as long as it is a commonly used route.

The administration route of the pharmaceutical composition according to the present invention may be oral or parenteral, and the parenteral administration may include oral, intravenous, intramuscular, intraarterial, intramedullary, intraarticular, intrasynovial, intrasternal, intrathecal, intracardiac, transdermal, subcutaneous, intradermal, intraperitoneal, intranasal, enteral, topical, intracranial, intracerebroventricular, intrauterine, intrauterine epidural, sublingual, or rectal administration, but is not limited thereto. The pharmaceutical composition of the present invention may be administered by any device through which the active ingredient may be transported to a target site.

The content of an active ingredient in the pharmaceutical composition may be appropriately adjusted according to the intended use of the pharmaceutical composition, the form of the formulation, and the lie, and may be, for example, 0.001% to 99% by weight, 0.001% to 90% by weight, 0.001% to 50% by weight, 0.01% to 50% by weight, 0.1% to 50% by weight, or 1% to 50% by weight based on the total weight of the pharmaceutical composition, but is not limited thereto.

In the present invention, the term ‘therapeutically effective amount’ means that the amount of the pharmaceutical composition administered alleviates one or more symptoms of a disorder being treated to some extent.

Therefore, a pharmacologically effective amount means an amount that has the effects of (1) reversing the rate of disease progression or, in the case of cancer, reducing the size of a tumor; (2) stopping the further progression of the disease to some extent or, in the case of cancer, slowing, or, preferably, stopping tumor metastasis to some extent; and/or (3) alleviating (preferably, eliminating) one or more symptoms associated with the disease to some extent.

The pharmaceutical composition according to the present invention contains active ingredients in an effective amount to achieve their intended purposes, and the dosage thereof may vary depending on various factors including the activity of the active ingredients, age, body weight, general health, sex, diet, administration time, administration route, excretion rate, drug combination, and the severity of a specific disease to be prevented or treated, and the dosage of the pharmaceutical composition may vary depending on the patient's conditions, body weight, degree of disease, drug form, and administration route and period, but may be appropriately selected by those skilled in the art, and the pharmaceutical composition may be administered in an amount of 0.0001 to 50 mg/kg or 0.001 to 50 mg/kg per day. Administration may be performed once a day or divided into several times. The above dosage does not limit the scope of the present invention in any way.

The pharmaceutical composition according to the present invention may be administered as an individual therapeutic agent or in combination with other therapeutic agents, may be administered sequentially or simultaneously with conventional therapeutic agents, and may be administered in a single dose or in multiple doses. Considering all of the above-described factors, it is important to administer an amount that may achieve the maximum effect with the minimum amount without side effects, and this may be easily determined by those skilled in the art to which the present invention pertains.

Hereinafter, the present invention will be described in more detail through examples and the like. However, the scope and content of the present invention should not be reduced or limited by the examples and the like. In addition, based on the disclosure of the present invention including the examples below, it is obvious that those skilled in the art may easily implement the present invention, even though specific experimental results are not presented, and it is obvious that such modifications and variations fall within the scope of the appended claims.

### <Experimental Materials>

Rink amide MBHA resin LL, 2-(6-chloro-1-H-benzotriazole-1-yl)-1,1,3,3-tetramethylammonium hexafluorophosphate (HCTU), 1-hydroxybenzotriazole (HOBt), and benzotriazole-1-yl-oxy-tris-pyrrolidino-phosphonium hexafluorophosphate (PyBOP) were purchased from Novabiochem (Germany). Fmoc-amino acids were purchased from Novabiochem (Germany), AnaSpec (USA), and AAPPTec (USA). 4% paraformaldehyde in phosphate-buffered saline (PBS) solution and 10% formalin were purchased from Biosesang (Republic of Korea). Matrigel matrix was purchased from Corning (USA). OCT (Optimal cutting temperature) compound was purchased from Scigen Scientific (USA). Sulfo-cyanine 5.5 NHS ester was purchased from Lumiprobe (Hong Kong). Antibodies were purchased from Santa Cruz Biotechnology (USA). SensoLyte® homogeneous AMC caspase-3/7 assay kit was purchased from AnaSpec (USA).

### Preparation Examples and Comparative Preparation Examples

### Peptide-Based Building Block

A peptide-based building block was synthesized using the Rink amide MBHA resin LL (resin substitution 0.36 mmol/g, synthesis scale 0.1 mmol) according to the standard Fmoc solid phase peptide synthesis (SPPS) protocol. The resin was preswollen with N-methyl-2-pyrrolidone (NMP) before the synthesis.

Fmoc amino acids were preactivated five minutes prior to the coupling reaction by mixing 4.5 eq. of HCTU and HOBt and 10 eq. of N,N-diisopropylethylamine (DIPEA) relative to the reaction scale in dimethylformamide (DMF). Each coupling step was performed for 30 minutes. Fmoc groups were deprotected with 20% piperidine in NMP for 15 minutes.

To generate an ester bond in the peptide backbone, 4 eq. of glycolic acid, 4 eq. of PyBOP, and 8 eq. of DIPEA were mixed in DMF/dichloromethane (DCM) (1:1) and allowed to react with the peptide for 90 minutes to couple glycolic acid to the N-terminus of the peptide. Next, the resulting production was treated with 10% hydrazine in DMF. After 30 minutes, a mixture of Fmoc amino acid (5 eq.), HOBt (5 eq.), DIPEA (10 eq.), and 4-(dimethylamino)pyridine (DMAP) (0.5 eq.) in DMF/DCM was added to the resin and allowed to react overnight to perform esterification. After the esterification, Fmoc amino acid coupling was performed. The above Fmoc reaction conditions were the same as the previous glycolic acid coupling, but the Fmoc deprotection reaction time was reduced to two minutes to prepare depsipeptides having the sequences of FIG. 2B (SEQ ID NO: 22 (Comparative Preparation Example 1), SEQ ID NOs: 23 to 32 (Production Preparations 1 to 10)) (2×). Preparation Examples 1 to 4 used W5 as a peptide for controlling drug release, Preparation Examples 5 and 6 used (WKWE) 2 as a peptide for controlling drug release, and in Preparation Example 5, a peptide for controlling drug release and a PPI sequence were linked via a linker (G). Preparation Example 7 used III and Preparation Example 8 used IIII as a peptide for controlling drug release, respectively, and Preparation Examples 7 to 10 used RRR as a cell-penetrating peptide. (GGS) 4, which was used as a PPI sequence in Preparation Example 7, was used as a type of linker to match the number of 12 amino acids.

TABLE 1

| SEQ ID | | |
|---|---|---|
| NO. | Name | Sequence (N->C) |
| 22 | Comparative Preparation | PRFWEYWLRLMEGR |
| | Example 1 | KKRRQRRR |
| | (MIP-Tat) | |
| 23 | Preparation Example 1 | WWWWWPRFWEYWLR |
| | (W5-MIP-Tat) | LMEGRKKRRQRRR |
| 24 | Preparation Example 2 | WWWWWPRFWEYWLR |
| | (W5-MIP-RGD) | LMEGRGDRGDRGD |
| 25 | Preparation Example 3 | WWWWWGGSEGGSEG |
| | (W5-dummy-Tat) | GSEGRKKRRQRRR |
| 26 | Preparation Example 4 | WWWWWGGSEGGSEG |
| | (W5-dummy-RGD) | GSEGRGDRGDRGD |
| 27 | Preparation Example 5 | WKWEWKWEGPRFWE |
| | ((WKWE)2-G-MIP-RGD) | YWLRLMEGRGDRGD |
| | | RGD |
| 28 | Preparation Example 6 | WKWEWKWEPRFWEY |
| | ((WKWE)2-MIP-Tat) | WLRLMEGRKKRRQR |
| | | RR |
| 29 | Preparation Example 7 | IIIGGSGGSGGSGG |
| | (III-(GGS)4-RRR) | SRRR |
| 30 | Preparation Example 8 | IIIIGGSGGSGGSG |
| | (IIII-(GGS)4-RRR) | GSRRR |
| 31 | Preparation Example 9 | WWWWWGGSGGSGGS |
| | (WWWWW-(GGS)4- | GGSRRR |
| | RRR) | |
| 32 | Preparation Example 10 | WKWEWKWEGGSGGS |
| | (WKWEWKWE-(GGS)4- | GGSGGSRRR |
| | RRR) | |

To separate the depsipeptide synthesized through the above-described process from the resin, the product was treated with a cleavage cocktail (trifluoroacetic acid/triisopropylsilane/water=92.5:2.5:2.5) at room temperature for four hours. The reaction product was treated with tert-butyl methyl ether (TBME).

The final product was purified by reversed-phase high-performance liquid chromatography (RP-HPLC) and analyzed using matrix-assisted laser desorption/ionization time-of-flight (MALDI-TOF) mass spectrometry, and the results are shown in FIG. 3.

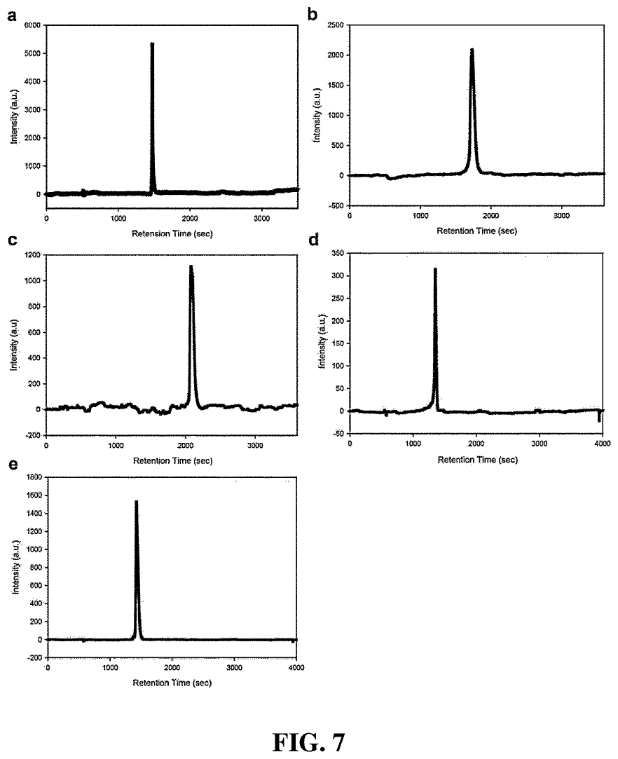

FIG. 3 shows MALDI-TOF MS spectra of purified peptide-based building blocks, and FIG. 7 shows HPLC chromatograms of purified peptide-based building blocks. FIG. 3A shows a depsipeptide-based building block of Comparative Preparation Example 1; FIG. 3B shows a depsipeptide-based building block of Preparation Example 1; FIG. 3C shows a depsipeptide-based building block of Preparation Example 2; FIG. 3D shows a depsipeptide-based building block of Preparation Example 3; and FIG. 3E shows a depsipeptide-based building block of Preparation Example 4. According to FIGS. 3 and 7, it can be seen that the peptide-based building blocks of Preparation Examples 1 to 4 and Comparative Preparation Example 1 were successfully synthesized and purified.

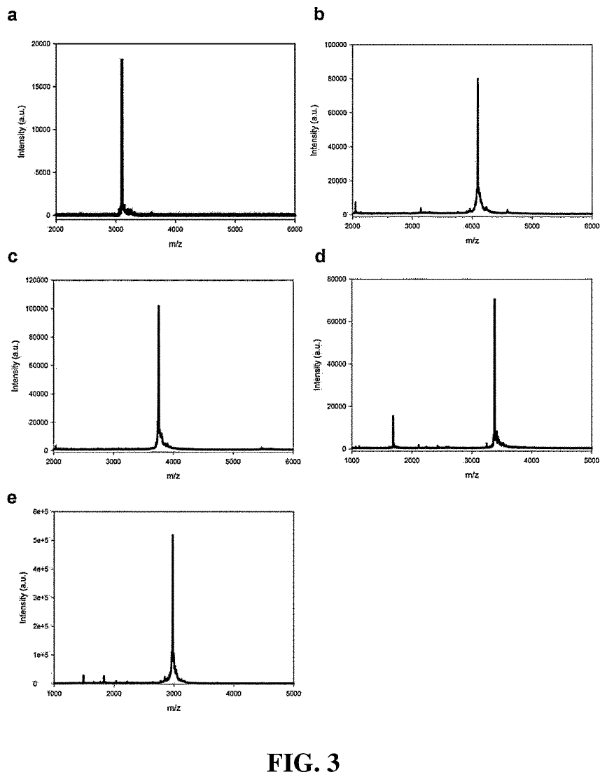

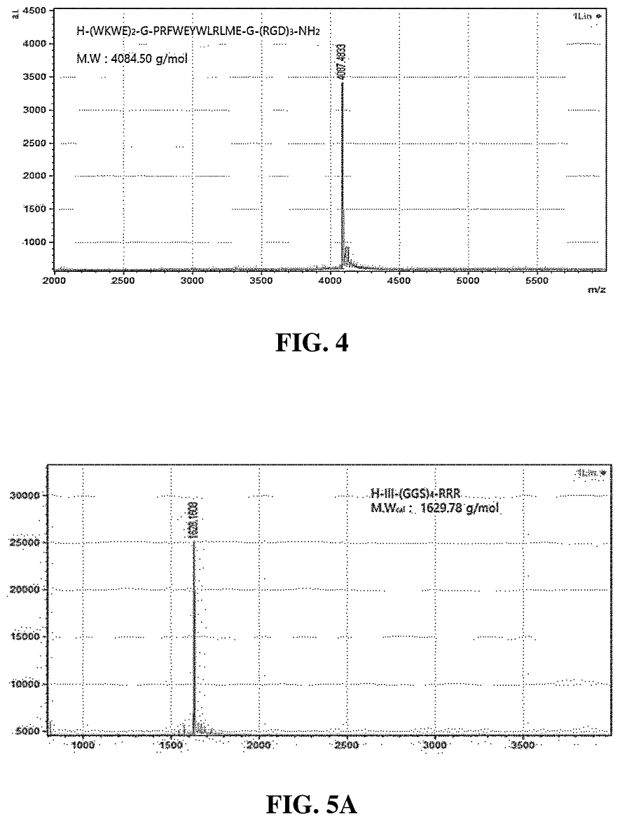

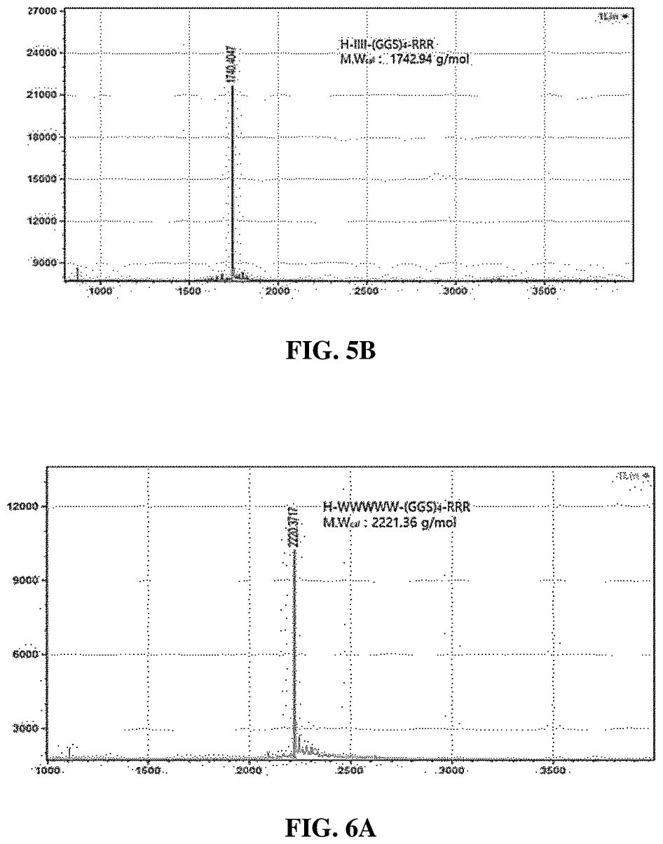

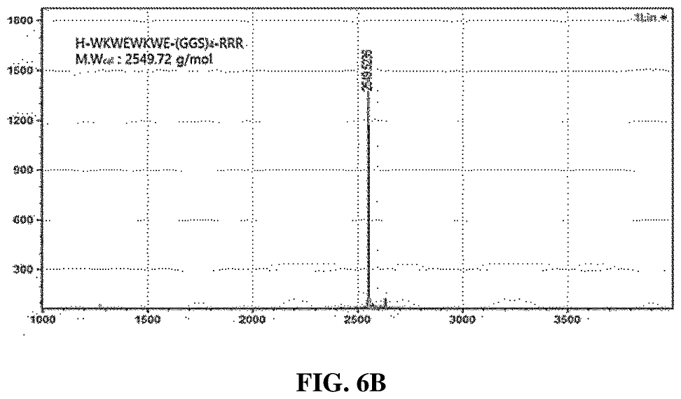

FIG. 4 shows a MALDI-TOF MS spectrum of the peptide-based building block of Preparation Example 5, FIG. 5A shows the spectrum of Preparation Example 7, FIG. 5B shows the spectrum of Preparation Example 8, FIG. 6A shows the spectrum of Preparation Example 9, and FIG. 6B shows the spectrum of Preparation Example 10. Through this, it can be seen that that the peptide-based building blocks of each preparation example were successfully synthesized and purified.

### <Examples 1 to 4 and Comparative Example 1> Preparation of SdPN (Biodegradable Self-Assembled Depsipeptide Nanostructures) Through Self-Assembly

The peptide-based building blocks prepared from Preparation Examples 1 to 4 and Comparative Preparation Example 1 were each dissolved in 80% ethanol and injected into an equal amount of PBS using a syringe to prepare each mixture. Each mixture was extruded through a 100 nm-sized polycarbonate membrane 10 times using an Avanti Extruder (Avanti Polar Lipids Inc., USA). Excess ethanol was removed using a SpeedVac vacuum concentrator to prepare each SdPN. The SdPN of Example 1 was prepared from the peptide-based building block of Preparation Example 1, the SdPN of Example 2 was prepared from the peptide-based building block of Preparation Example 2, the SdPN of Example 3 was prepared from the peptide-based building block of Preparation Example 3, the SdPN of Example 4 was prepared from the peptide-based building block of Preparation Example 4, and the SdPN of Comparative Example 1 was prepared from the peptide-based building block of Comparative Preparation Example 1.

Each SdPN was prepared in the same manner using the depsipeptide-based building blocks prepared from Preparation Examples 5 to 10.

### <Examples 5 to 12> Preparation of SdPN Through Molecular Co-Self-Assembly

The depsipeptide-based building blocks prepared from Preparation Examples 1 to 4 were each dissolved in 80% ethanol, and the two depsipeptide-based building blocks were injected into PBS at various molar ratios using a syringe to prepare mixtures (Table 2). Each mixture was extruded through a 100 nm-sized polycarbonate membrane 10 times using an Avanti Extruder (Avanti Polar Lipids Inc., USA). Excess ethanol was removed using a SpeedVac vacuum concentrator to prepare each SdPN.

SdPN was prepared in the same manner according to the proportion of the depsipeptides of Preparation Examples 5 and 6 (prepared by varying the molar ratios of Preparation Examples 5 and 6 to 9:1, 7.5:2.5, 5:5, 2.5:7.5, and 1:9).

TABLE 2

| | Molar ratio | | | Molar ratio | |
|---|---|---|---|---|---|
| | Preparation | Preparation | | Preparation | Preparation |
| | Example 1 | Example 2 | | Example 3 | Example 4 |
| | W5-MIP-Tat | W5-MIP-RGD | | W5-dummy-Tat | W5-dummy-RGD |
| Example 5 | 7.5 | 2.5 | Example 11 | 5 | 5 |
| Example 6 | 7 | 3 | | | |
| Example 7 | 6.5 | 3.5 | | | |
| Example 8 | 6 | 4 | Example 12 | 6 | 4 |
| Example 9 | 5.5 | 4.5 | | | |
| Example 10 | 5 | 5 | | | |

### <Experimental Example 1> Structure of Depsipeptide-Based Building Block

The peptide-based building block (MIP-Tat) of Comparative Preparation Example 1 and the depsipeptide-based building block (W5-MIP-Tat) of Preparation Example 1 were each put into an aqueous solution and measured at 190 to 250 nm at 25° C. using a Chirascan circular dichroism (CD) spectrometer (Applied Photophysics Inc., UK).

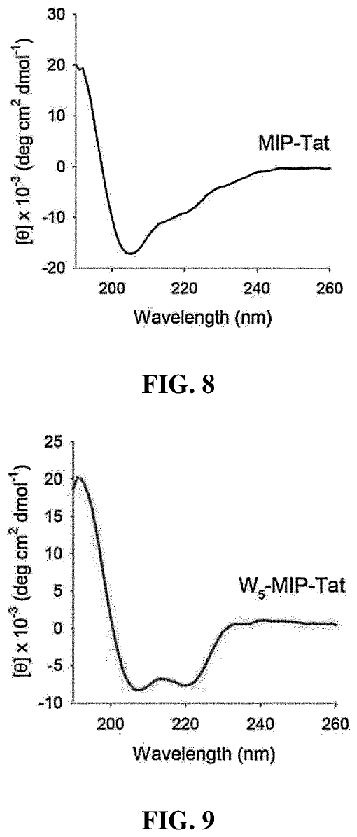

FIG. 8 shows a CD spectrum of the peptide-based building block (MIP-Tat) of Comparative Preparation Example 1, and FIG. 9 shows a CD spectrum of the depsipeptide-based building block (W5-MIP-Tat) of Preparation Example 1.

As shown in FIGS. 8 and 9, that peptide-based building blocks formed nanostructures through self-assembly in an aqueous solution at room temperature, and in this case, the secondary structure of each peptide could be confirmed.

It was confirmed that the peptide-based building block (MIP-Tat) of Comparative Preparation Example 1 had a random coil structure without a regular secondary structure.

On the other hand, it was confirmed that the depsipeptide-based building block (W5-MIP-Tat) of Preparation Example 1 had an α-helical structure (α-helical state). It is believed that the depsipeptide-based building block (W5-MIP-Tat) of Preparation Example 1 underwent a secondary structural transformation from a random ring state to an α-helical state due to adjacent peptide molecules when forming a nanostructure through self-assembly. Therefore, the depsipeptide-based building block (W5-MIP-Tat) of Preparation Example 1 may maintain the α-helical secondary structure without losing it even when forming a self-assembled nanostructure, SdPN, in an aqueous solution due to the peptide of SEQ ID NO: 1.

The peptide for inhibiting PPIs used in the present invention is an MIP ligand (SEQ ID NO: 14), which is an MDM2/MDMX-binding peptide. This inhibits the interaction of MDM2/MDMX, thereby activating the function of the p53 protein, inhibiting the growth of cancer cells, and promoting the tumor suppression mechanism. At this time, since the MIP ligand (SEQ ID NO: 14) is known to bind to MDM2 in an α-helical state, it can be seen that the depsipeptide-based building block according to the present invention maintains the secondary structure of a peptide for inhibiting/promoting PPIs, such as the MIP ligand, so that the efficacy is maintained in vivo.

### <Experimental Example 2> Cell Culture and Cytotoxicity Analysis

The cancer cell toxicity of the peptide-based building block according to the present invention was confirmed by the WST-8 assay. First, HCT116 cells (human colon cancer cells) were cultured in a thermos-hygrostat incubator at 37° C. and 5% CO₂ in Dulbecco's modified Eagle's Medium (DMEM) containing 10% fetal bovine serum (FBS) and 1% Pen-Step.

For a WST-8 cell viability assay, HCT116 cells were seeded at a density of 2×10⁴ cells/well in 100 mL culture medium in a 96-well plate and cultured for 24 hours. Each Opti-MEM reduced serum medium (Gibco Inc., USA) containing 0, 5, 10, 20, and 40 μM samples (SdPN of Example 1, SdPN of Example 2, SdPN of Example 3, and co-self-assembled SdPN of Examples 10 and 11) was prepared (sample medium), and the cell culture medium was replaced with each sample medium. After culturing at 37° C. for 12 to 24 hours, WST-8 reagent (Abcam Inc., UK) was added to each well, and culture was performed for 30 minutes. The absorbance at 570 nm was measured using a microplate reader (PerkinElmer Inc., USA). At this time, the conventional anticancer drug Nutlin-3a was used as a control group. Nutlin-3a is well known as an inhibitor suppressing the p53-MDM2 interaction.

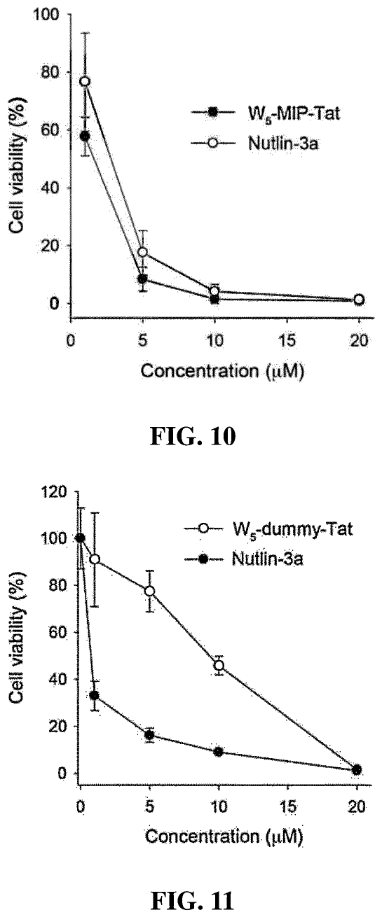

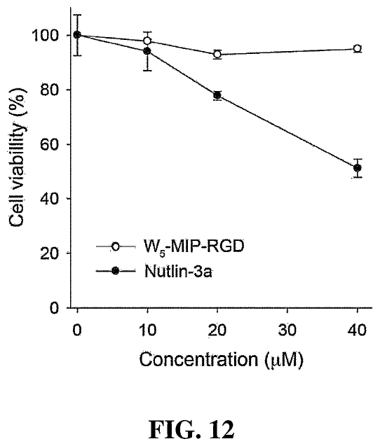

FIG. 10 shows a graph of analyzing the viability of HCT116 cells for SdPN and Nutlin-3a prepared according to Example 1 at concentrations of 0, 5, 10, and 20 μM, FIG. 11 shows a graph of analyzing the viability of HCT116 cells for the SdPN prepared according to Example 3 and Nutlin-3a at concentrations of 0, 5, 10, and 20 μM, FIG. 12 shows a graph of analyzing the viability of HCT116 cells for the SdPN prepared according to Example 3 and Nutlin-3a at concentrations of 0, 10, 20, and 40 μM. Data are shown as mean±standard deviation (n=3).

As shown in FIG. 10, it was confirmed that the SdPN prepared according to Example 1 had an excellent cancer cell apoptotic effect not only at low concentrations but also at high concentrations. It was confirmed that the SdPN of Example 1 according to the present invention induces cell apoptosis by inhibiting MDM2 and restoring the p53 pathway.

However, nanostructures of which surface was modified with multiple positively charged moieties such as arginine induce necrosis by damaging cell membrane integrity. In order to distinguish between apoptosis and necrosis, the SdPN prepared according to Example 3, including a dummy sequence of a random sequence (SEQ ID NO: 14), was designed.

The SdPN of Example 3 is a peptide consisting of 12 random sequences replacing the MIP ligand. As shown in FIG. 11, it was confirmed that the SdPN prepared according to Example 3 exhibited a cancer cell apoptotic effect, although the effect was lower than that of the SdPN of Example 1. The SdPN of Example 3 was non-specific because it lacked a MIP ligand, but it exhibited cytotoxicity, confirming that necrosis cytotoxicity was induced.

It was confirmed that the SdPN of Example 1 and SdPN of Example 3 both exhibited cytotoxicity against cancer cells, although their cell death mechanisms of were different due to the different types of (b) ligands.

On the other hand, as shown in FIG. 12, it can be confirmed that the SdPN of Example 2 exhibited almost no cytotoxicity. RGD, a cell-penetrating peptide, is a zwitterionic sequence with neutralized positive charges and does not cause damage to the cell membrane. In addition, it was confirmed that the SdPN of Example 2 has an advantage in that it may remain in the cell for a long time without being degraded or released by endosomes and lysosomes after being absorbed into the cell through the RGD-integrin interaction.

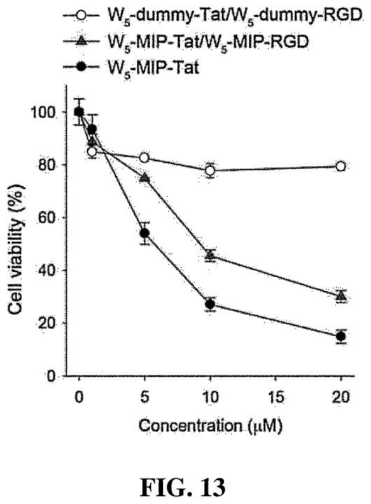

Based on the above-described results, it was expected that the SdPN of Example 1 and the SdPN of Example 2 would complement each other to reduce necrosis and increase cell invasion and endosome escape. Accordingly, the co-self-assembled SdPN of Example 10 (W5-MIP-Tat/W5-MIP-RGD=5:5) and the co-self-assembled SdPN of Example 11 (W5-dummy-Tat/W5-dummy-RGD=5:5) were prepared, and their cell viability was analyzed (FIG. 13).

FIG. 13 shows a graph analyzing the viability of HCT116 cells for co-self-assembled SdPN (W5-MIP-Tat/W5-MIP-RGD=5:5) prepared according to Example 10, co-self-assembled SdPN (W5-dummy-Tat/W5-dummy-RGD=5:5) prepared according to Example 11, and Nutlin-3a at concentrations of 0, 5, 10, and 20 μM. Data are shown as mean±standard deviation (n=3).

As shown in FIG. 13, the co-self-assembled SdPN (W5-MIP-Tat/W5-MIP-RGD-5:5) of Example 10 exhibited a significant level of cytotoxicity, whereas the co-self-assembled SdPN (W5-dummy-Tat/W5-dummy-RGD=5:5) prepared according to Example 11 exhibited almost no cytotoxicity. In other words, it can be seen that the co-assembly of depsipeptide-based building blocks prepared with different cell-penetrating peptides reduced the side effect of non-specific necrotic cytotoxicity due to the W5-ligand-cell-penetrating peptide structure excluding the MIP ligand and regulated the cytotoxicity to be induced only through the apoptosis pathway.

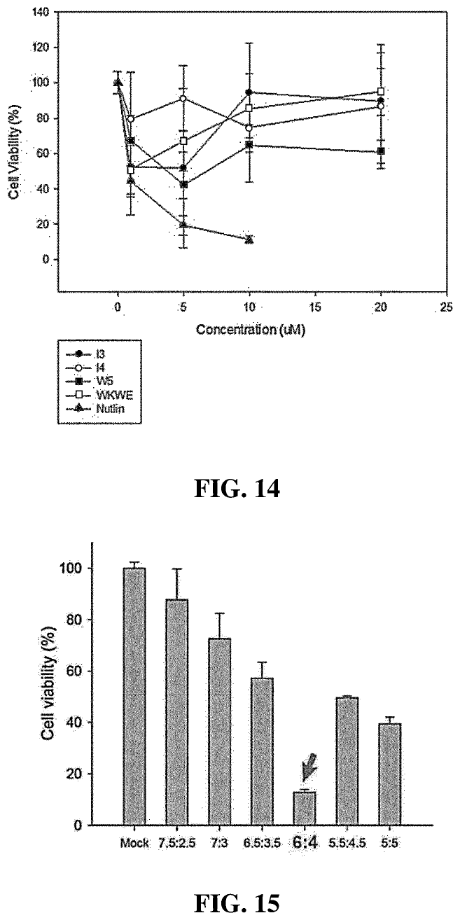

In addition, as shown in FIG. 14, the cytotoxicity of the depsipeptide-based SdPN prepared according to the sequences (SEQ ID Nos: 1 to 4) of the peptide for controlling drug release (wherein the sequence of the peptide for inhibiting PPIs was PRFWEYWLRLME and the cell-penetrating peptide sequence was Tat sequence) was confirmed at concentrations of 0, 5, 10, 15, and 20 μM. As a result, it was confirmed that the depsipeptide-based SdPN using W5 as a peptide for controlling drug release exhibited a significant cytotoxic effect compared to the peptides for controlling drug release including other sequences, thereby confirming the efficacy of the W5 peptide.

### <Experimental Example 3> Analysis of Cytotoxicity According to Molar Ratio of Two Depsipeptide-Based Building Blocks

Cytotoxicity was analyzed by performing an experiment in the same manner as in Experimental Example 2, except that the co-self-assembled SdPNs of Examples 5 to 10 (W5-MIP-Tat/W5-MIP-RGD-7.5 to 5:5 to 2.5) were each used as samples, and the results are shown in FIG. 15.

FIG. 15 shows a graph analyzing the viability of HCT116 cells for co-self-assembled SdPNs of Examples 5 to 10 (W5-MIP-Tat/W5-MIP-RGD=7.5 to 5:5 to 2.5). Data are shown as mean±standard deviation (n=3).

As shown in FIG. 15, in order to precisely optimize the proportion of the two depsipeptide-based building blocks, co-self-assembled SdPNs were prepared at various molar ratios, and their cytotoxicity was evaluated. As a result, it was confirmed that the co-self-assembled SdPN of Example 8 (W5-MIP-Tat/W5-MIP-RGD-6:4) had the best cytotoxicity.

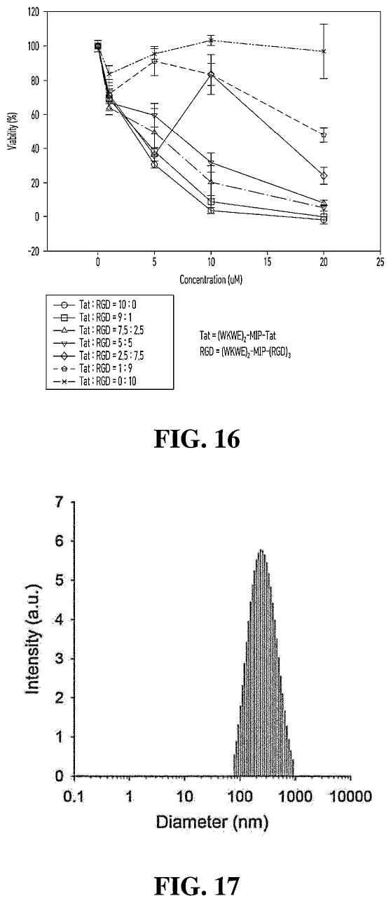

As shown in FIG. 16, the cytotoxicity of SdPN according to the proportion of the depsipeptides of Preparation Examples 5 and 6 was confirmed, and as a result, it was found that when the SdPN included only Preparation Example 5 ((WKWE)2-G-PRFWEYWLRLME-G-(RGD)3), there was no cytotoxicity, and when the SdPN included only Preparation Example 6 ((WKWE)2-PRFWEYWLRLME-G-RKKRRQRRR(Tat)), the cytotoxicity was strong, and that the cytotoxicity varied depending on the proportion of the two Preparation Examples.

In other words, it was confirmed that the peptide for controlling drug release according to the present invention forms a nanostructure through self-assembly without destroying the secondary structure of a ligand (e.g. MIP ligand) that inhibits/promotes PPIs.

Based on the above-described technology, for the intracellular delivery of a ligand, a depsipeptide-based building block consisting of a peptide for controlling drug release, a ligand, and a cell-penetrating peptide was designed, and it was confirmed that the building blocks effectively formed a nanostructure (SdPN) through self-assembly in a solution phase at room temperature and induced apoptosis by penetrating into cancer cells upon contact.

The SdPN according to the present invention has an excellent anticancer effect on cancer cells, but nonspecific necrosis is induced depending on the type of cell-penetrating peptide. Therefore, in order to minimize this, depsipeptide-based building blocks having different cell-penetrating peptides were co-self-assembled to minimize nonspecific necrosis while having an excellent anticancer effect.

In particular, the co-self-assembled SdPN of Example 8 (W5-MIP-Tat/W5-MIP-RGD=6:4) was confirmed to have the best cancer cell apoptotic effect through MDM2 inhibition while minimizing non-specific necrotic cytotoxicity by minimizing cell membrane destabilization.

### <Experimental Example 4> Analysis of Structural Features of SdPN (6:4) Prepared from Example 8

The nanostructural features of the SdPN (6:4) prepared from Example 8 were investigated. The SdPN (6:4) (50 μM) prepared from Example 8 was added to PBS (10 mM potassium phosphate, 150 mM NaCl, pH 6.3), and dynamic light scattering (DLS) measurement was performed using an ELS-1000ZS particle size analyzer (Otsuka Electronics CO., LTD., Japan) at 25° C. to determine the average size distribution of SdPN (6:4). Next, in order to analyze the thermal stability, a Chirascan CD spectrometer (Applied Photophysics Inc., UK) was used to measure at 190 to 250 nm at various temperatures (10, 20, 30, 40, 50, 60, 70° C.) or storage periods (1 to 4 days, 25° C.).

The SdPN (6:4) prepared from Example 8 was analyzed by atomic force microscopy (AFM). The sample was gently dropped onto a freshly cleaved mica surface, incubated at room temperature for one minute, washed with distilled water, and the remaining water droplets were removed with a filter paper, and then the resulting product was completely dried at room temperature before measurement. AFM was performed in a non-contact mode using a Park NX10 instrument (Park Systems Co., Ltd., Republic of Korea) at a scan rate of 0.3 Hz and a Z-gain of 1.

For transmission electron microscopy (TEM) measurement, 1 μL of the sample was deposited on a carbon-coated copper grid and completely dried. The grid was washed with 1 mL of distilled water to remove salt crystals. The sample was negatively stained with 2% (w/v) uranyl acetate, and TEM measurement was performed with a JEM-F200 multipurpose analytical S/TEM (JEOL Co. Ltd., Japan).

FIG. 17 shows a graph illustrating the DLS analysis results of the SdPN (6:4) prepared from Example 8, and according to the results, it can be confirmed that the SdPN (6:4) prepared from Example 8 had an average diameter of 219.9±1.0 nm. Since the SdPN (6:4) prepared from Example 8 had a diameter of 100 to 300 nm at a physiological salt concentration (˜150 mM NaCl), it is useful for intracellular and in vivo delivery.

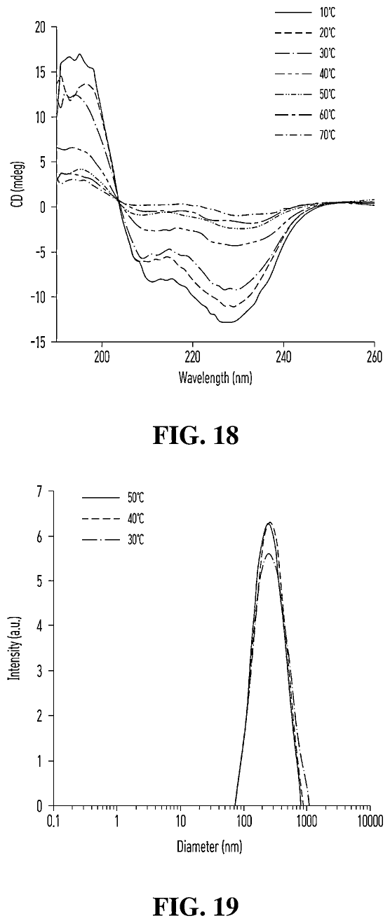

FIG. 18 shows a graph illustrating the CD analysis results of SdPN (6:4) prepared from Example 8 under various temperature conditions. According to the results, it was confirmed that the α-helix structure of the SdPN (6:4) prepared from Example 8 had significant thermal stability.

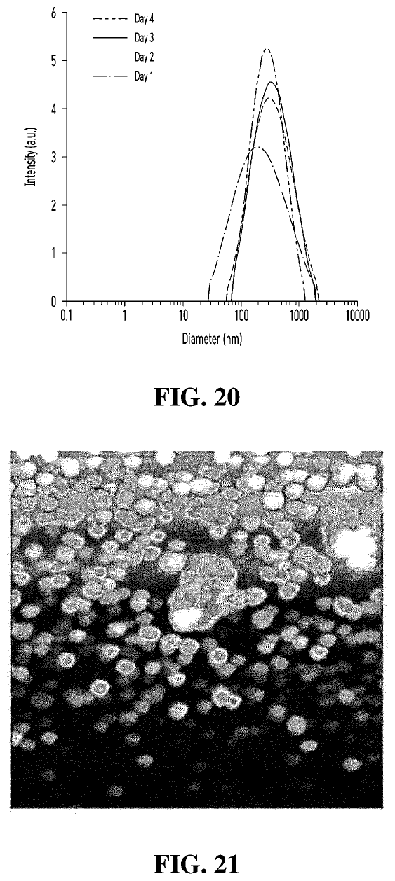

FIG. 19 shows a graph analyzing the DLS analysis results of SdPN (6:4) prepared from Example 8 under various temperature conditions, and FIG. 20 shows a graph analyzing the DLS analysis results of SdPN (6:4) prepared from Example 8 according to various storage periods.

As shown in FIGS. 19 and 20, it was confirmed that the SdPN (6:4) of Example 8 maintained its size distribution even at a temperature higher than the body temperature (˜37° C.). In addition, it was confirmed that the SdPN (6:4) of Example 8 maintained its size distribution even during a storage period of one to four days, indicating that the SdPN according to the present invention has excellent thermal stability and long-term storage properties.

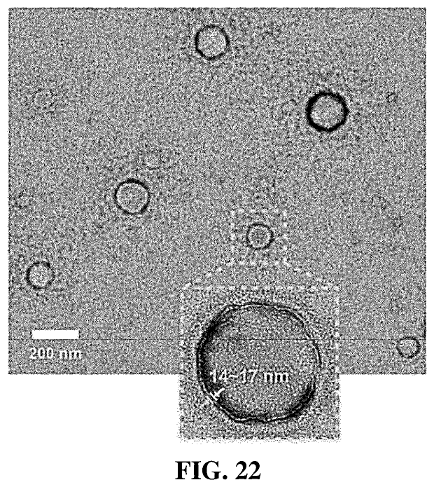

FIG. 21 shows an atomic force microscopy (AFM) image (5×5 μm in size) of SdPN (6:4) prepared from Example 88, and FIG. 22 shows a transmission electron microscopy (TEM) image of SdPN (6:4) prepared from Example 8.

As shown in FIGS. 21 and 22, it can be confirmed that the SdPN (6:4) prepared from Example 8 was a spherical particle in the form of a vesicle including a single bilayer membrane. The thickness of the bilayer membrane was 14 to 17 nm, which means that the length of the depsipeptide-based building block was 7 to 9 nm, and thus it can be seen that the SdPN was successfully formed by two depsipeptide-based building blocks.

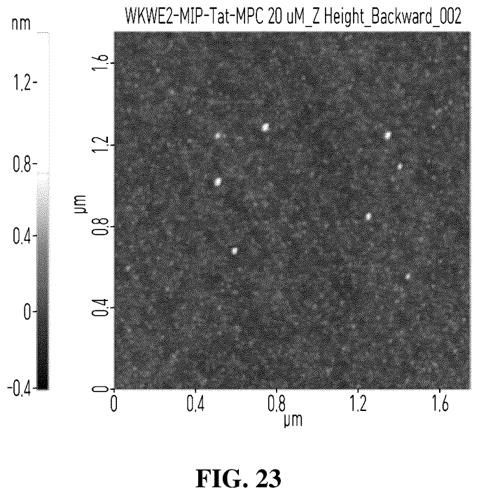

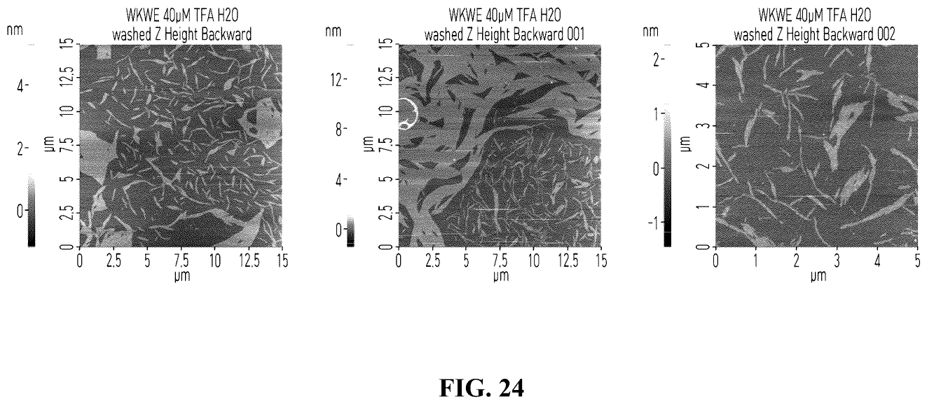

FIG. 23 shows an AFM image of depsipeptide-based SdPN of Preparation Example 6, and FIG. 24 shows an AFM image of depsipeptide-based SdPN of Preparation Example 10.

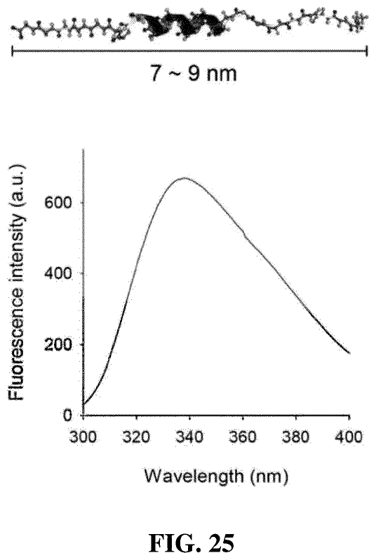

FIG. 25 shows a fluorescence emission spectrum (λₑₓ=280 nm) of SdPN (6:4) prepared from Example 8. The maximum emission of tryptophan fluorescence was blueshifted to 338 nm in the SdPN (6:4) prepared from Example 8. This means that the W5 peptide (SEQ ID NO: 1) is located in the hydrophobic region of the bilayer membrane in the vesicle structure of SdPN (6:4).

### <Experimental Example 5> Analysis of the Cancer Cell Apoptotic Effect of SdPN (6:4) Prepared from Example 8 and Small Molecules

The anticancer effects of the SdPN (6:4) prepared from Example 8 and idasanutlin were compared and analyzed in HCT116 p53^{+/+} cells. Idasanutlin is a second-generation anticancer drug that blocks the interaction between MDM2 and p53, and is most actively used in human studies, so it was used as a comparative group.

First, HCT116 p53^{+/+} cells were cultured in a thermos-hygrostat incubator at 37° C., 5% CO₂ in DMEM containing 10% FBS and 1% Pen-Step.

For a WST-8 cell viability assay, the HCT116 p53+/+ cells were seeded at a density of 2×10⁴ cells/well in 100 mL culture medium in a 96-well plate and cultured for 24 hours. Each Opti-MEM reduced serum medium (Gibco Inc., USA) containing 0, 5, 10, 15, and 20 μM samples (co-self-assembled SdPN of Example 8 and idasanutlin) was prepared (sample medium), and the cell culture medium was replaced with each sample medium. After culturing at 37° C. for 12 to 24 hours, WST-8 reagent (Abcam Inc., UK) was added to each well, and culture was performed for 30 minutes. The absorbance at 570 nm was measured using a microplate reader (PerkinElmer Inc., USA).

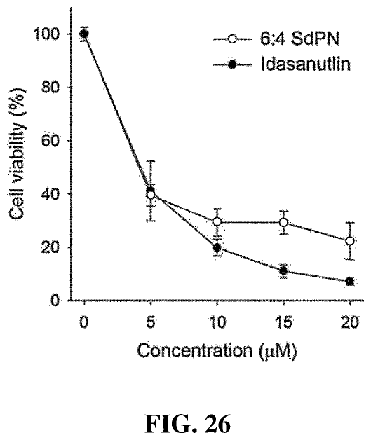

FIG. 26 shows a graph analyzing the viability of HCT116 p53^{+/+} cells treated with the SdPN (6:4) prepared from Example 8 and idasanutlin. According to the results, it was confirmed that the SdPN (6:4) prepared from Example 8 had a cancer cell apoptotic effect at a level similar to that of the comparative group, idasanutlin. Data were shown as mean±standard deviation (n=4).

### <Experimental Example 6> Comparison of PPI-Inhibiting Effects of SdPN (6:4) Prepared from Example 8 and Small Molecules

The anticancer effects of the SdPN (6:4) prepared from Example 8 and idasanutlin were compared and analyzed in HCT116 p53+/+ cells. Idasanutlin was used as a comparative group.

HCT116 p53+/+ cells were seeded in a 6-well plate and cultured, and then each well was treated with 10 μM idasanutlin or 10 μM SdPN (6:4) prepared from Example 8, and then the cells were cultured for various periods of time (0, 3, 6, 9, 12, 15, 18, 21, 24 h). The cells were harvested, washed with cold PBS, and lysed with a radio-immunoprecipitation assay (RIPA) buffer. The cell lysate was centrifuged at 4° C. and 13,000 rpm for 20 minutes, and the supernatant was collected. The protein amount of the lysate was quantified using a bicinchoninic acid (BCA) protein assay kit (Pierce Inc., USA). The supernatant of the cell lysate was loaded on 10% sodium dodecyl sulfate-polyacrylamide gel electrophoresis (SDS-PAGE) to separate proteins, which were transferred to a polyvinylidene fluoride (PVDF) membrane, and the membrane was treated with 5% skim milk in tris-buffered saline with Tween® 20 (TBS-T) buffer (room temperature), and after one hour, washed with TBS-T buffer (2×) for one minute. After treatment with primary antibodies (specific primary antibodies) (p53, MDM2, or vinculin), the membrane was incubated overnight at 4° C. The membrane was washed with TBS-T for 10 minutes (5×), treated with horseradish peroxidase (HRP)-conjugated secondary antibodies (room temperature, 1 h), and then washed with TBS-T for 10 minutes (5×) and analyzed using a LAS 3000 luminescent image analyzer (Fujifilm Co., LTD., Japan).

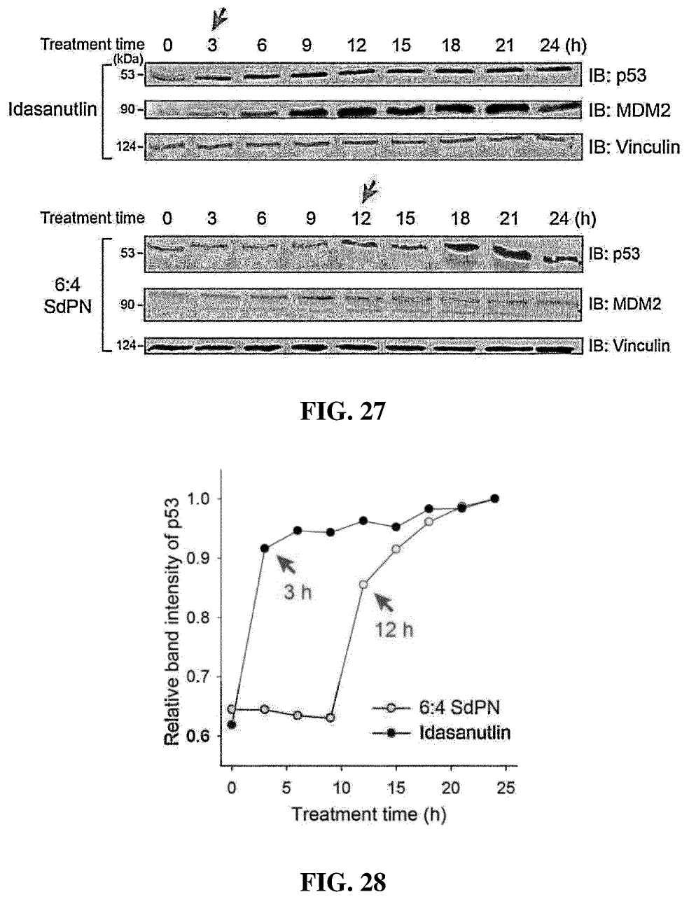

FIG. 27 shows the results of analyzing the reaction behavior (kinetic analysis) of SdPN (6:4) prepared from Example 8 and idasanutlin on MDM2 and p53 analyzed by immunoblotting, and FIG. 28 shows a graph illustrating the quantification of the p53 expression level from the immunoblotting results of FIG. 27.

As shown in FIGS. 27 and 28, the cancer cell apoptosis by the SdPN (6:4) prepared from Example 8 was confirmed by the gradual increase of the p53 expression level over time, indicating that the cell apoptotic effect of SdPN (6:4) prepared from Example 8 was caused by inducing p53 reactivation in cancer cells, as in the case of idasanutlin.

It was confirmed that the SdPN (6:4) prepared from Example 8 took 12 hours to increase the level of p53 expression in cells.

### <Experimental Example 7> Hydrolysis of Ester Bond of SdPN (6:4) Prepared from Example 8

Porcine liver esterase purchased from Sigma-Aldrich (USA) was used for the experiment.

Various concentrations of porcine liver esterase (0, 40, 60, 80, 100, 120 units/mL) were mixed in 10 mM potassium phosphate (PPB, pH 7.4), and the SdPN (6:4) (20 mM) prepared from Example 8 was added thereto. The mixtures were incubated at 37° C. for 12 hours to prepare hydrolysates. The prepared hydrolysates were analyzed by RP-HPLC. The analysis process was performed using a C4 HPLC column equipped with a guard column (XBridge protein BEH C4 semi-prep column, 10 mm×250 mm, 300 Å, 5 mm), and the solvent (mobile phase) used was a mixed solution of solvent A (water (0.1% trifluoroacetic acid (TFA))) and solvent B (acetonitrile (0.1% TFA)). At this time, a concentration gradient method was employed in which the concentration of solvent B in the mixed solution of solvent A and solvent B was gradually increased from 5% to 65%, and the measurement was performed at 25° C. for 30 minutes at a flow rate of 2 mL/min. The data were analyzed through integration of peaks detected at 230 nm. The peak area of each hydrolysate was normalized by comparing it with the peak area in the non-esterified state.

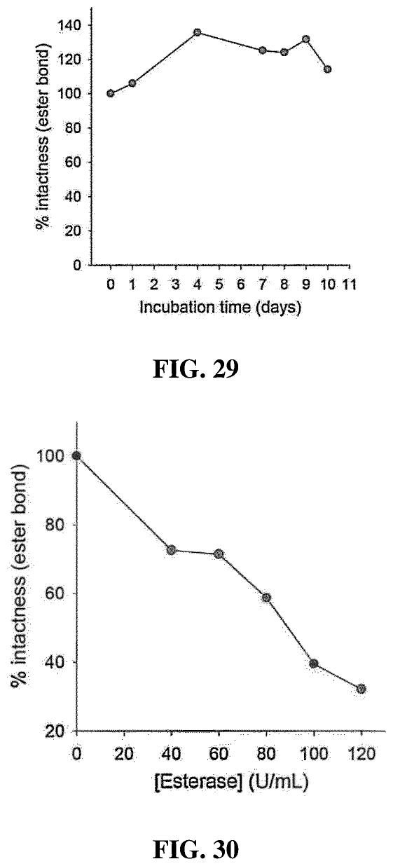

FIG. 29 shows a graph illustrating the degree of ester bond degradation of depsipeptide-based building blocks (W5-MIP-Tat and W5-MIP-RGD) analyzed by HPLC when SdPN (6:4) prepared from Example 8 was incubated under conditions in which an esterase was absent, and FIG. 30 shows a graph illustrating the degree of ester bond degradation of depsipeptide-based building blocks (W5-MIP-Tat and W5-MIP-RGD) analyzed by HPLC when SdPN (6:4) prepared from Example 8 was incubated under esterase conditions of various concentrations.

As shown in FIGS. 29 and 30, the ester bond of the SdPN (6:4) prepared from Example 8 was hardly degraded in vitro in the absence of esterase. However, it was confirmed that the SdPN (6:4) prepared from Example 8 was degraded in a dose-dependent manner under conditions in which esterase was present.

Since cancer cells have a high level of cytosolic esterase activity, the SdPN (6:4) prepared from Example 8 was degraded by the esterase to effectively release the MIP ligand having a cancer cell apoptotic effect, which means that the release and release time of the MIP ligand having an anticancer effect can be effectively controlled (release mechanism). Although the conventional anticancer drug idasanutlin exhibited a fast PPI inhibition action speed of three hours, the action speed was too fast to exhibit sufficient efficacy when considering the in vivo absorption, the time for the drug to reach the cell, and the like. However, the SdPN according to the present invention (particularly the SdPN (6:4) prepared from Example 8) has significant and remarkable characteristics in the sense that sufficient arrival time and absorption time into cancer cells can be secured, and the drug effect onset time can be controlled even in the context of combined administration with other anticancer drugs, so sequential cancer apoptosis and suppression effects may be exhibited or a continuous anticancer effect may be obtained.

### <Experimental Example 8> Caspase-3/7 Activity Analysis

First, HCT116 p53^{+/+} cells were cultured in a thermos-hygrostat incubator at 37° C. and 5% CO₂ in DMEM containing 10% FBS and 1% Pen-Step. The HCT116 p53^{+/+} cells were treated with SdPN (6:4) of Example 8, dummy-SdPN (6:4) of Example 12, and idasanutlin, and analyzed using SensoLyte homogeneous AMC caspase-3/7 assay kit (AnaSpec Inc., USA). Untreated cells were used as a control group.

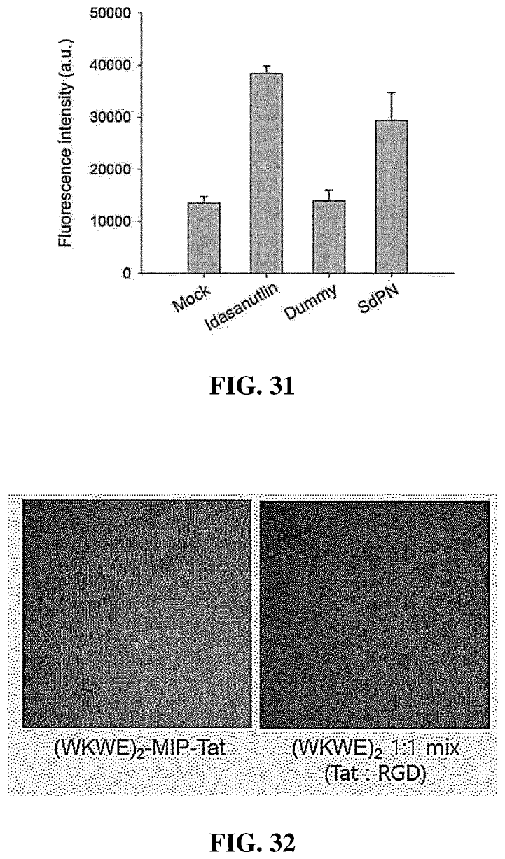

FIG. 31 shows the results of a Caspase-3/7 activity analysis for SdPN (6:4) of Example 8, dummy-SdPN (6:4) of Example 12, and idasanutlin. According to the results, it was confirmed that the cancer cell apoptotic effect of SdPN (6:4) of Example 8 also induced an increase in Caspase-3/7 activity due to p53 activation. In other words, it can be seen that the SdPN (6:4) of Example 8 caused an increase in the activity of the two proteins, thereby achieving excellent cancer cell removal and apoptotic effects.

FIG. 32 shows the results of a Caspase-3/7 activity analysis for the depsipeptide-based nanostructure of Preparation Example 6 and a depsipeptide-based structure prepared by mixing the depsipeptides of Preparation Examples 5 and 6 at a ratio of 1:1. It was confirmed that p53 activation induced an increase in the Caspase-3/7 activity.

### <Experimental Example 9> Cellular Thermal Shift Assay (CETSA) Analysis

For CETSA, the cultured HCT116 cells were washed with PBS, and after replacing the culture solution with Opti-MEM, the cells were cultured for one hour. The culture solution was treated with 10 μM of SdPN (6:4) of Example 8 or 10 μM of dummy-SdPN (6:4) of Example 11, cultured for 12 hours, and then treated with a phosphatase-protease inhibitor cocktail. The treated cells were heated at each temperature (40° C., 43.4° C., 46.9° C., 50.3° C., 57.1° C., 60.6° C.) for three minutes, and then proteins were extracted, and the expression of MDM2, p53, and β-actin was analyzed by SDS-PAGE and immunoblotting.

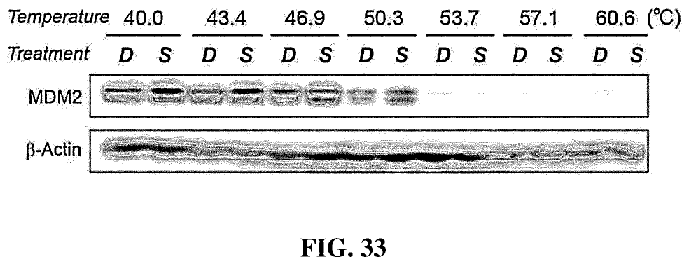

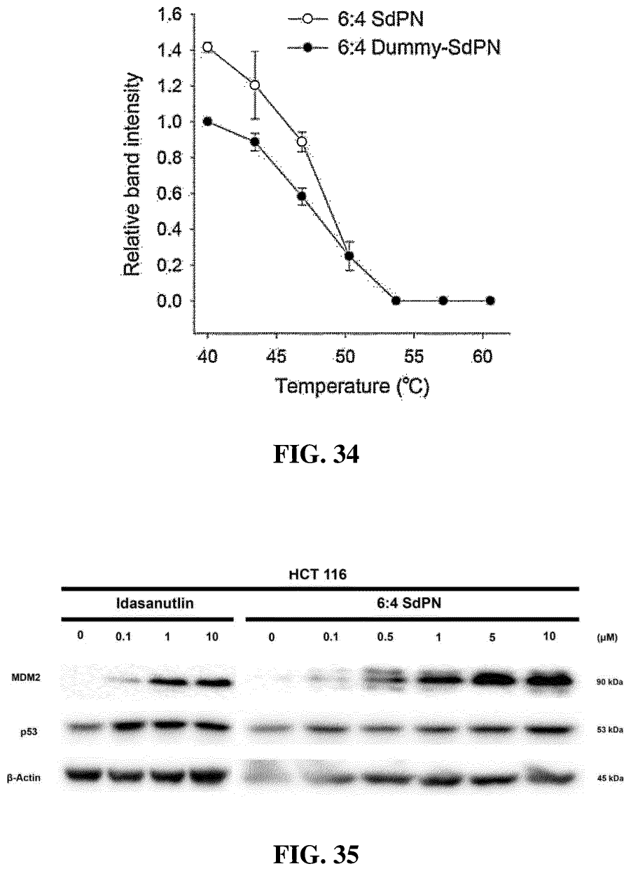

FIG. 33 shows the results of a CETSA for SdPN (6:4) of Example 8 and dummy-SdPN (6:4) of Example 11, and FIG. 34 shows a graph illustrating the quantitative analysis of the CETSA results for analyzing the Tₘ (melting temperature) of SdPN (6:4) of Example 8 and dummy-SdPN (6:4) of Example 11. Data are expressed as mean±standard deviation (n=3). In FIGS. 33 and 34, ‘D’ indicates the experiment where the cells were treated with dummy-SdPN (6:4) of Example 11, and ‘S’ indicates the experiment where the cells were treated with SdPN (6:4) of Example 8.

As shown in FIGS. 33 and 34, it was confirmed that the SdPN (6:4) of Example 8 increased the thermal stability of MDM2. It was also confirmed that the dummy-SdPN (6:4) of Example 11 had thermal stability.

However, it was confirmed that the Tₘ of SdPN (6:4) of Example 8 was 2.8° C. higher than the Tₘ of dummy-SdPN (6:4) of Example 11, indicating that SdPN (6:4) of Example 8 exhibited better specificity in vivo.

### <Experimental Example 10> Iso-CETSA Analysis

For iso-CETSA (isothermal dose-response assay), the cultured HCT116 cells were prepared, washed with PBS, and then cultured in Opti-MEM culture solution for one hour. The cells were treated with SdPN (6:4) prepared in Example 8 or idasanutlin at various concentrations (0, 0.1, 0.5, 1, 5, 10 μM). After culturing for six hours, each sample was treated with a phosphatase-protease inhibitor cocktail and heated to 46° C. After cooling to room temperature for two minutes, the samples were placed on ice and stored.

To each sample of the above stored cells, PBS containing 0.4% NP-40 lysis buffer was added, and two freezing/thawing lysis cycles were performed in liquid nitrogen. Insoluble proteins were removed by centrifugation, and the supernatant containing soluble proteins was recovered, and the expression of MDM2, p53, and β-actin expression was analyzed by SDS-PAGE and immunoblotting.

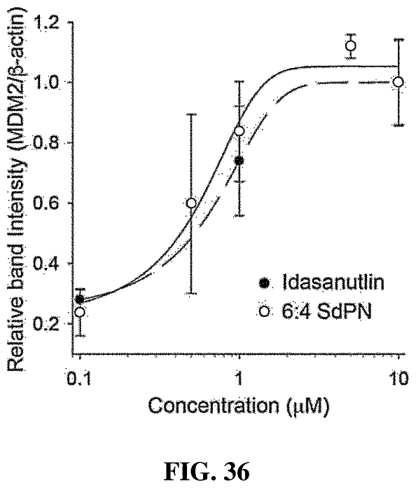

FIG. 35 shows the iso-CETSA results of SdPN (6:4) prepared from Example 8 and idasanutlin, and FIG. 36 shows a graph illustrating the IC50 level obtained from the iso-CETSA results of SdPN (6:4) prepared from Example 8 or idasanutlin. Data are expressed as mean±standard deviation (n=4).

As shown in FIGS. 35 and 36, it was confirmed that the SdPN (6:4) of Example 8 increased the thermal stability of MDM2 in a dose-dependent manner, similar to idasanutlin.

However, it was confirmed that the SdPN (6:4) of Example 8 exhibited a significantly lower IC50 than idasanutlin (1.4 μM and 3.7 μM, respectively). This means that the SdPN (6:4) of Example 8 has a 3-fold higher affinity for MDM2 than idasanutlin in vivo.

### <Experimental Example 11> Analysis of In Vivo Anticancer Effects

In order to verify the anticancer efficacy of the SdPN according to the present invention, an analysis was performed using a cancer animal model (xenograft) in which human cancer cells were cultured in immunodeficient mice. Specifically, a mixture of 50 mL of Matrigel (Corning, NY, USA) and HCT116 cells (5×10⁶ cells in 50 mL of saline) was subcutaneously injected into the hind legs of 5-week-old nu/nu male nude mice (nu/nu athymic). After three days, the SdPN (6:4) of Example 8 (experimental group, n=4), idasanutlin (positive control group, n=4), and saline (control group, n=4) were intravenously injected through the tail vein of the xenografted mice. The dose was 76.9 nmol/100 μl saline of the SdPN (6:4) of Example 8 or idasanutlin, and the administration was performed once every two days for a total of five times. Body weight and tumor volume of each group were measured every 0, 2, 4, 6, 8, 10, 12, and 14 days.

After 14 days, cancer tissues and major organs (heart, lung, liver, spleen, and kidney) were isolated from each group, preserved in 10% formalin, sectioned, and hematoxylin and eosin (H&E) stained to obtain images using an AxioImager Al optical microscope (Carl Zeiss Co., LTD., Germany).

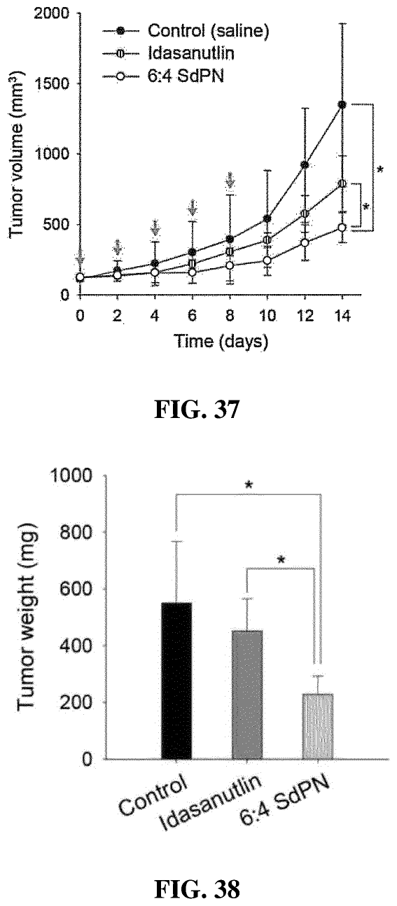

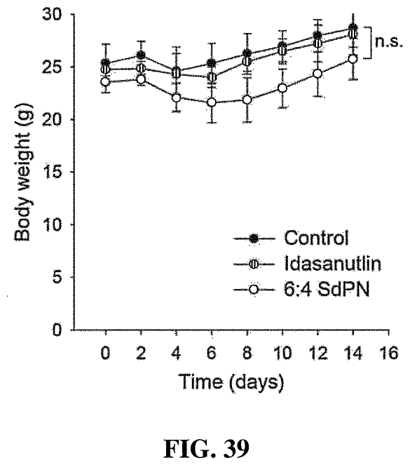

FIG. 37 shows a graph illustrating the change in tumor volume of xenografted mice measured after the intravenous administration of SdPN (6:4) (experimental group) of Example 8, idasanutlin (positive control group), and saline (control group) (mean±standard deviation, *P<0.05); FIG. 38 shows a graph illustrating the tumor weight of xenografted mice 14 days after the intravenous administration of SdPN (6:4) (experimental group) of Example 8, idasanutlin (positive control group), and saline (control group) (mean±standard deviation, *P<0.05); and FIG. 39 shows a graph illustrating the change in body weight of xenografted mice measured after the intravenous administration of SdPN (6:4) (experimental group) of Example 8, idasanutlin (positive control group), and saline (control group) (mean±standard deviation, ‘n.s.’ indicates no significant difference. *P<0.05).

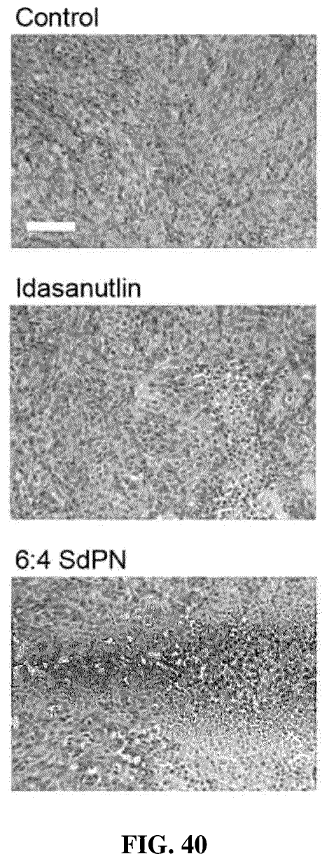

FIG. 40 shows the results of H&E staining of cancer tissues isolated from xenografted mice 14 days the intravenous administration of SdPN (6:4) (experimental group) of Example 8, idasanutlin (positive control group), and saline (control group) (bar=50 μm).

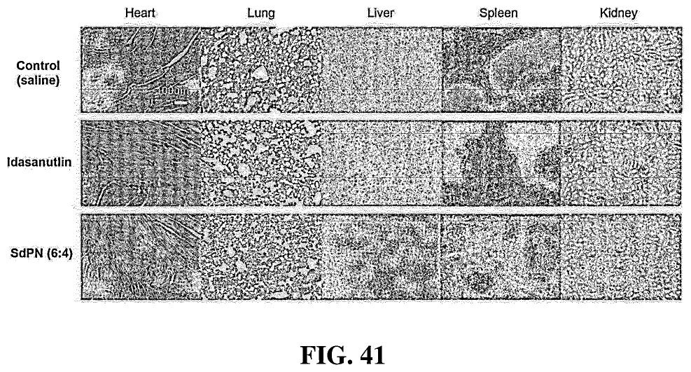

FIG. 41 shows the results of H&E staining of major organ tissues (heart, lung, liver, spleen, and kidney) isolated from xenografted mice 14 days after the intravenous administration of SdPN (6:4) (experimental group) of Example 8, idasanutlin (positive control group), and saline (control group) (bar=100 μm).

As shown in FIGS. 37 to 41, it was confirmed that cancer growth was significantly inhibited in the mice treated with idasanutlin and the SdPN (6:4) of Example 8. Furthermore, the tumor volume and tumor weight of the mice treated with the SdPN (6:4) of Example 8 were significantly reduced more than those treated with idasanutlin (about 1.5 to 2 times), which means that the anticancer effect of the SdPN (6:4) of Example 8 is superior to that of conventional anticancer agents in vivo.

In addition, the mice treated with the SdPN (6:4) of Example 8 exhibited no significant change in body weight, confirming that it had no harmful effects on the body.

As shown in FIG. 40, it was confirmed that the cancer tissues of the mouse treated with the SdPN (6:4) of Example 8 were most severely damaged.

On the other hand, as shown in FIG. 41, no significant damage was observed in the major organ tissues of the mice treated with SdPN (6:4) of Example 8. In other words, it can be confirmed that the SdPN (6:4) of Example 8 has a significantly superior anticancer effect to conventional anticancer agents, without serious side effects.

Although it was confirmed in the cell experiment that idasanutlin exhibited a higher anticancer effect than that of the SdPN (6:4) of Example 8, it was confirmed that the SdPN (6:4) of Example 8 had a better anticancer effect in vivo. It can be seen that the SdPN (6:4) of Example 8 penetrates cancer cells better, accumulates better, and has better cancer cell specificity than small molecule anticancer agents such as idasanutlin.

### <Experimental Example 12> Confirmation of Cancer Cell-Specific Effect

In order to verify the tumor specificity of the SdPN according to the present invention, an analysis was performed using a cancer animal model (xenograft) in which human cancer cells were cultured in immunodeficient mice. Specifically, a mixture of 50 mL of Matrigel (Corning, NY, USA) and HCT116 cells (5× 10⁶ cells in 50 mL of saline) was subcutaneously injected into the hind legs of 5-week-old nu/nu male nude mice (nu/nu athymic). The tumor volume was analyzed using a caliper [tumor volume=(major axis)×(minor axis)²/2]. When the tumor volume reached 250 mm², the SdPN (6:4) (SdPN-dye) of Example 8 labeled with sulfo-cyanine 5.5 (Cy5.5) or Cy5.5 (free dye) was intravenously injected through the tail vein of the xenografted mouse (n=3). At this time, the dose was 100 μl of saline injected at a concentration of 400 μM based on Cy5.5 (40 nmol/100 μl saline).

Fluorescence images were obtained at various times after the intravenous injection. To confirm the biodistribution, the mice were sacrificed after 24 hours, and tumor tissues and major organs (heart, lung, liver, spleen, and kidney) were separated, and then fluorescence images were obtained. Fluorescence images were obtained using an in vivo imaging system (IVIS) (Lumia XRMS, PerkinElmer Inc., USA) with a Cy5.5 filter (ex: 500, em: 710 nm).

The isolated cancer tissues were treated with 4% paraformaldehyde, and their moisture content was assessed with sucrose. After being embedded in an optimal cutting temperature (OCT) compound, the tissues were stored at −80° C. overnight, cryosectioned using a cryocut microtome (Leica Biosystems GmbH, Germany), stained with Hoechst 33342, and photographed with an inverted fluorescence microscope (IX71, Olympus CO., LTD., Japan) to obtain fluorescence images.

The SdPN (6:4) of Example 8 labeled with Cy5.5 (SdPN-dye) was allowed to react with 2 molar equivalents of Cy5.5 NHS ester, which is an amine-reactive dye (relative to the primary amine functional group of the peptide), with the SdPN (6:4) of Example 8. At this time, the SdPN (6:4) of Example 8 was mixed in 10 mM potassium phosphate, pH 7.4, containing 20% glycerol and used. The reactant of Cy5.5 and the SdPN (6:4) of Example 8 was allowed to react overnight under dark conditions, and then excess dye was removed through a dialysis membrane (3,500 MWCO), and then the product was analyzed by MALDI-TOF MS, fluorescence emission spectrum, and DLS.

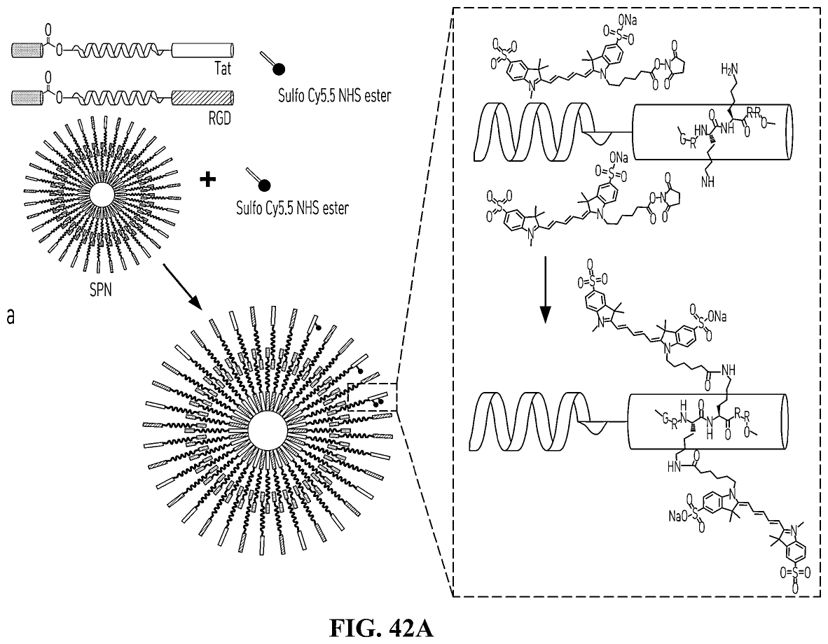

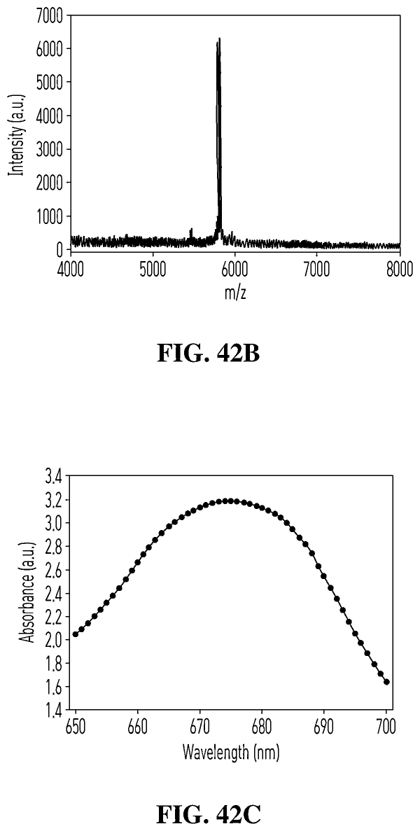

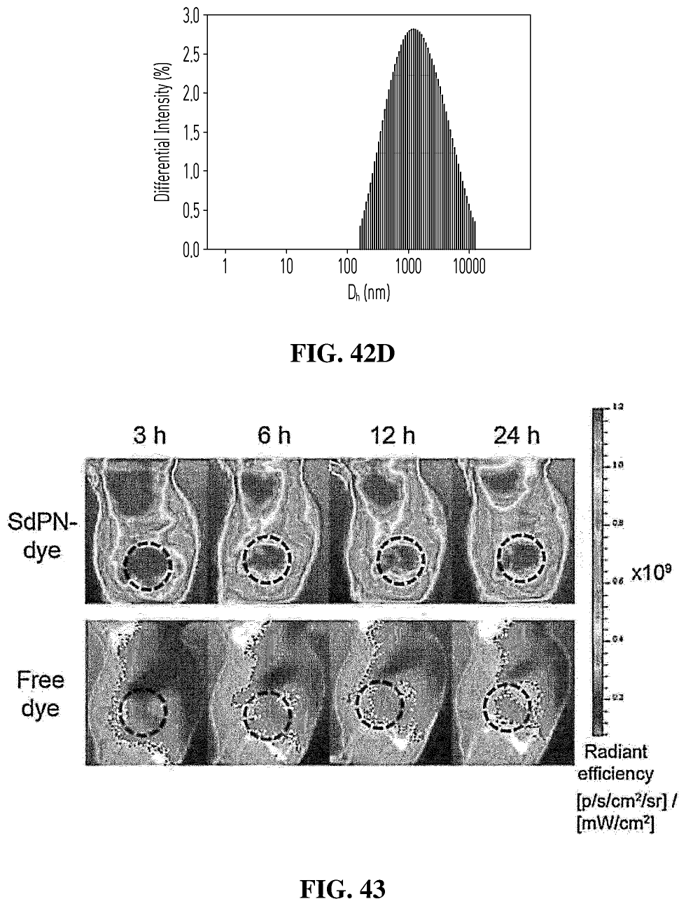

FIGS. 42A-42D respectively show the synthesis process (FIG. 42A), MALDI-TOF MS (FIG. 42B), fluorescence emission spectrum (FIG. 42C), and DLS analysis results (FIG. 42D) of SdPN (6:4) (SdPN-dye) of Example 8 labeled with Cy5.5.

As shown in FIGS. 42A-42D, it was confirmed that the SdPN (6:4) of Example 8 was successfully coupled with the fluorescent probe sulfo-Cy5.5, indicating that a uniform nanostructure could be obtained without aggregation, although the average diameter increased to some extent.

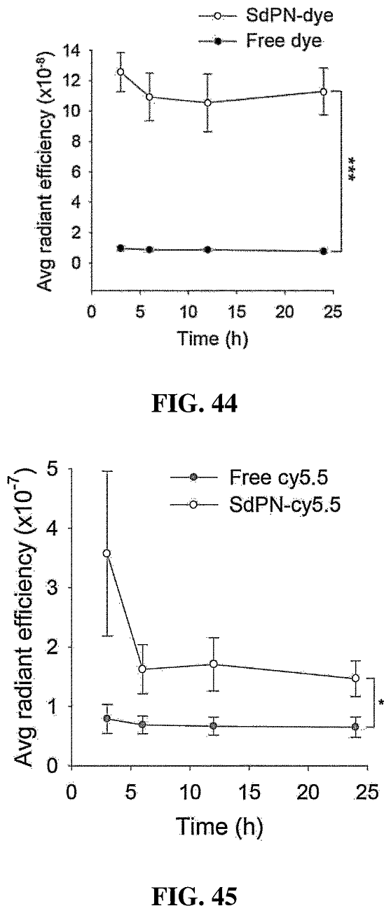

FIG. 43 shows in vivo fluorescence images (IVIS) of xenografted mice treated with Cy5.5-labled SdPN (6:4) (SdPN-dye) of Example 8 and Cy5.5. The cancer area is indicated by a dotted line on the figure. FIG. 44 shows a graph illustrating the fluorescence intensity quantified from the IVIS image data of FIG. 43 (mean±standard deviation, n=4, ***P<0.001).

As shown in FIGS. 43 and 44, the biodistribution was traced in the xenografted mice. As a result, it was confirmed that in the group treated with SdPN (6:4) (SdPN-dye) of Example 8, the drug accumulated in a large amount (15 times compared to Cy5.5) in the cancer tissue area for a long of time. On the other hand, it was confirmed that small molecules such as Cy5.5 not only failed to be specifically localized to the cancer area but also were rapidly removed over time.

FIG. 45 shows a graph illustrating the fluorescence intensity measured in the blood of xenografted mice treated with Cy5.5-labled SdPN (6:4) (SdPN-dye) of Example 8 and Cy5.5.

As shown in FIG. 45, it was confirmed that the SdPN (6:4) of Example 8 was present at a low concentration in blood vessels.

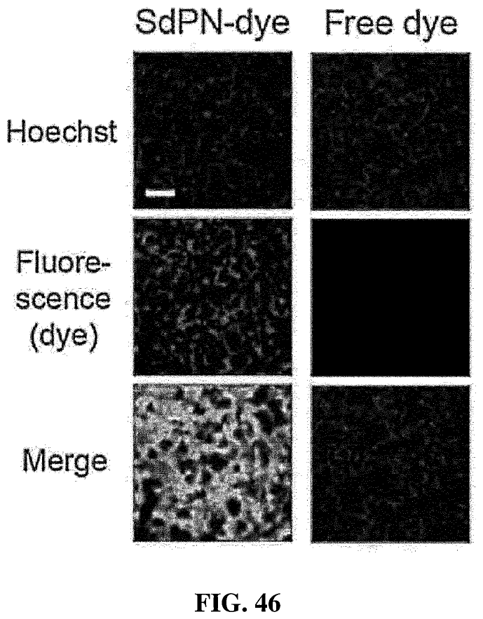

FIG. 46 shows fluorescence images of frozen sections of cancer tissues isolated from xenografted mice 24 hours after the intravenous administration of Cy5.5-labled SdPN (6:4) (SdPN-dye) of Example 8 and Cy5.5 (bar=50 μm).

As shown in FIG. 46, it was confirmed that the SdPN (6:4) of Example 8 effectively penetrated into cancer tissues, especially cancer cells, and accumulated in large quantities, whereas no significant accumulation was confirmed for Cy5.5.

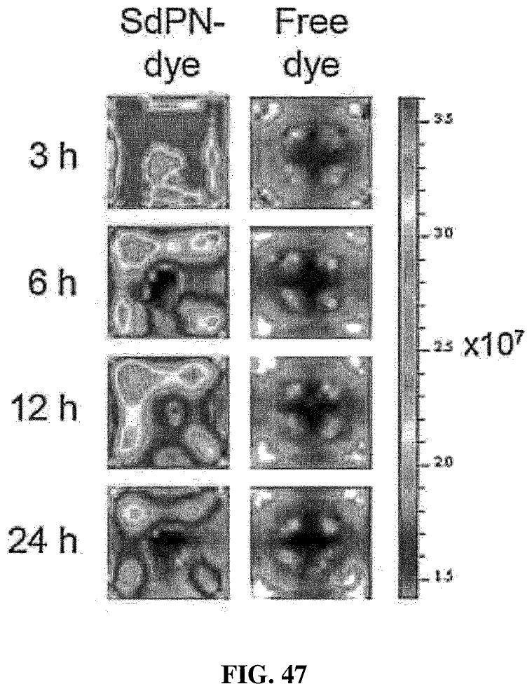

FIG. 47 shows a fluorescence image of tail vein blood obtained from xenografted mice intravenously administered with Cy5.5-labled SdPN (6:4) (SdPN-dye) of Example 8 and Cy5.5 (bar=50 μm).

As shown in FIG. 47, the SdPN (6:4) of Example 8 gradually decreased over time after the intravenous administration, whereas small molecules such as Cy5.5 were completely eliminated within a short time. It was confirmed that the in vivo retention period of the SdPN (6:4) of Example 8 was significantly higher.

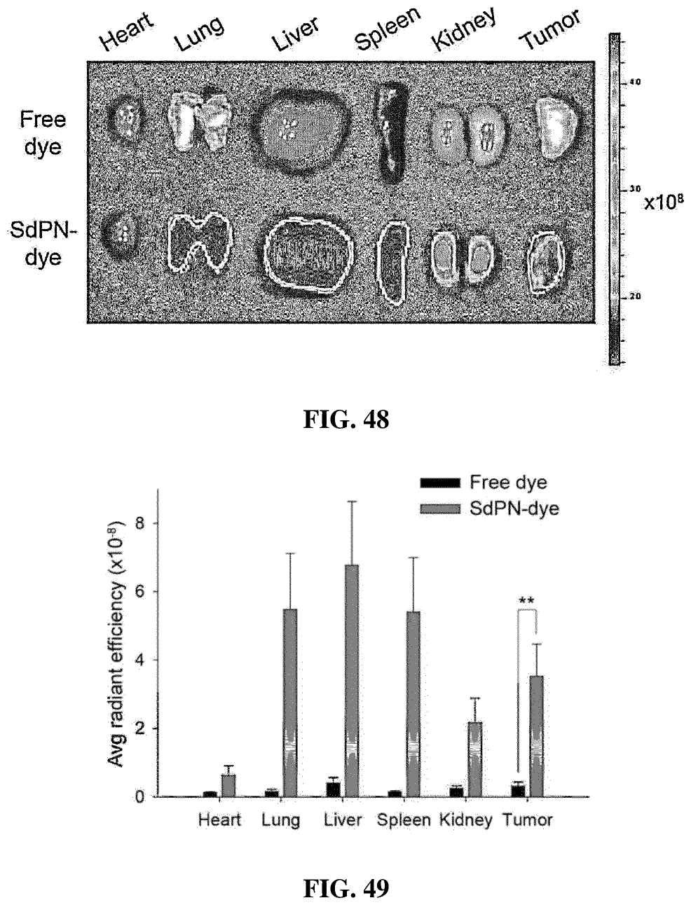

FIG. 48 shows fluorescence images of major organs (heart, lung, liver, spleen, and kidney) and cancer tissues isolated from a xenograft mouse 24 hours after the intravenous administration of Cy5.5-labled SdPN (6:4) (SdPN-dye) of Example 8 and Cy5.5, and FIG. 49 shows a graph the fluorescence intensity quantified from the fluorescence image data of FIG. 48 (mean±standard deviation, n=4, ***P<0.001).

As shown in FIGS. 48 and 49, it was confirmed that the SdPN (6:4) of Example 8 accumulated in the lungs, liver, spleen, and kidneys for a long time, but did not cause damage (no death or weight change). Small molecules such as Cy5.5 exhibited significant accumulation neither in major organ tissues nor cancer tissues.

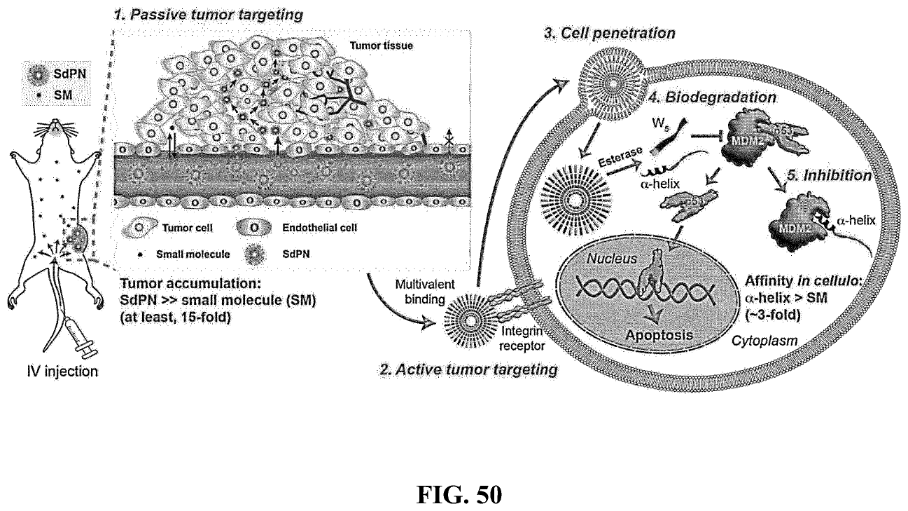

FIG. 50 illustrates the mechanism by which the depsipeptide-based nanostructure according to the present invention forms a nanostructure in a solution phase and acts on cancer cells when injected into the body.

As shown in FIG. 50, when the SdPN according to the present invention is injected into a body through the blood, it penetrates the blood vessel wall, forms multiple bonds with the intergrin receptors present on the surface of cancer cells, penetrates into the cells, and is degraded by an esterase present in the cells to release the MIP ligand. The released MIP ligand binds to MDM2, thereby inhibiting the binding of MDM2 and p53. Through this, the MIP ligand induces the reactivation of p53, thereby inducing cancer cell apoptosis. Even when the SdPN according to the present invention is formed into a nanostructure through self-assembly, it maintains the secondary structure of the MIP ligand, which is an α-helix form, without being deformed or collapsed in the body, thereby maintaining the affinity for MDM2 three times higher than that of small molecule anticancer agents in cancer cells.

The present invention can provide a new platform that enables easy and unrestricted use in vivo of the numerous PPI-inhibiting peptide drugs developed through drug screening or peptide library screening techniques while maintaining their efficacy.

The depsipeptide-based building block of the present invention forms a nanostructure through self-assembly in a solution phase, and when administered in vivo, the depsipeptide-based building block can remain in the body stably for a long time to deliver a peptide for inhibiting PPIs to a target tissue and gradually release a peptide for inhibiting PPIs over a long time to obtain a high effect.

In addition, since the present invention has excellent thermal stability and has superior pharmacodynamic properties than small molecule drugs in vivo, various applications are possible in the field of treatment using peptides inhibiting protein-protein interactions.

What is claimed is:

1. A peptide for controlling drug release for a cancer target, the peptide represented by any one selected from the group consisting of SEQ ID NOs: 1 to 4, wherein hydrogen positioned at a hydroxyl group at the C-terminus of the peptide is substituted with a carboxyl group having 1 to 5 carbon atoms.

2. The peptide for controlling drug release according to claim 1, wherein the peptide for controlling drug release has hydrophobicity and a function of inhibiting deformation of a secondary structure when combined with a peptide having an α-helix secondary structure.

3. A depsipeptide-based building block consisting of:
(a) the peptide for controlling drug release according to claim 1;
(b) a peptide for inhibiting protein-protein interactions (PPIs) with an α-helix secondary structure consisting of 12 amino acid residues; and
(c) a cell-penetrating peptide;
wherein a C-terminal of the peptide for controlling drug release and an N-terminal of the peptide for inhibiting PPIs are connected by an ester bond (—C(O)O—).

4. The depsipeptide-based building block according to claim 3, wherein (b) the peptide for inhibiting PPIs is a peptide having an activity of inhibiting the interaction between mouse double minute 2 homolog (MDM2) protein or murine double minute X homolog (MDMX) and p53 protein.

5. The depsipeptide-based building block according to claim 3, wherein (b) the peptide for inhibiting PPIs is a fragment of an α-helix peptide of p53 represented by SEQ ID NO: 13.

6. The depsipeptide-based building block according to claim 3, wherein (b) the peptide for inhibiting PPIs is any one selected from SEQ ID NOs: 14 to 18.

7. The depsipeptide-based building block according to claim 3, wherein the (c) cell-penetrating peptide is linked to a C-terminus of (b) the peptide for inhibiting PPIs by a peptide bond.

8. The depsipeptide-based building block according to claim 3, wherein the cell penetrating peptide is represented by any one selected from the group consisting of SEQ ID NOs: 19 to 21.

9. The depsipeptide-based building block according to claim 3, wherein the total length of the depsipeptide-based building block is 7 to 9 nm on average.

10. The depsipeptide-based building block according to claim 3, wherein (b) the peptide for inhibiting PPIs and the (c) cell-penetrating peptide are linked via a linker.

11. The depsipeptide-based building block according to claim 10, wherein the linker is one or more selected from Gly, Gly-Gly, and Gly-Gly-Gly.

12. A nanostructure of a spherical shape having a bilayer membrane formed through self-assembly, comprising the depsipeptide-based building block according to claim 3.

13. The nanostructure according to claim 12, wherein the depsipeptide-based building block is any one of the depsipeptide-based building blocks represented by SEQ ID NOs: 5 to 12, or a mixture thereof.

14. The nanostructure according to claim 13, wherein the depsipeptide-based building block mixture is a mixture of a depsipeptide-based building block represented by SEQ ID NO: 5 and a depsipeptide-based building block represented by SEQ ID NO: 6 at a molar ratio of 6:4.

15. The nanostructure according to claim 13, wherein the average diameter of the nanostructure is 100 to 500 nm.

16. A pharmaceutical composition for treating or preventing cancer, comprising the nanostructure according to claim 13 as an active ingredient

17. The pharmaceutical composition for treating or preventing cancer according to claim 16, wherein the cancer is one or more selected from the group consisting of brain tumor, head and neck cancer, breast cancer, lung cancer, esophageal cancer, stomach cancer, duodenal cancer, appendix cancer, colon cancer, rectal cancer, liver cancer, pancreatic cancer, gallbladder cancer, bile duct cancer, anal cancer, kidney cancer, ureter cancer, bladder cancer, prostate cancer, penile cancer, testicular cancer, uterine cancer, ovarian cancer, vulvar cancer, vaginal cancer, or skin cancer.
